(19) United States
(12) Patent Application Publication
(10) Pub. No.: US 2014/0039044 A1
(43) Pub. Date: Feb. 6, 2014

(54) Hydroxylated Polymethoxyflavone Compositions

(71) Applicants: Chi-Tang Ho, East Brunswick, NJ (US); Shiming Li, Glastonbury, CT (US); Min-Hsiung Pan, Kaohsiung City (TW); Chih-Yu Lo, Somerset, NJ (US); Slavik Dushenkov, Fort Lee, NJ (US)

(72) Inventors: Chi-Tang Ho, East Brunswick, NJ (US); Shiming Li, Glastonbury, CT (US); Min-Hsiung Pan, Kaohsiung City (TW); Chih-Yu Lo, Somerset, NJ (US); Slavik Dushenkov, Fort Lee, NJ (US)

(73) Assignees: WLLGEN, INC., New Brunswick, NJ (US); RUTGERS, THE STATE UNIVERSITY OF NEW JERSEY, New Brunswick, NJ (US)

(21) Appl. No.: 13/935,991

(22) Filed: Jul. 5, 2013

Related U.S. Application Data
(63) Continuation of application No. 11/724,558, filed on Mar. 14, 2007, now abandoned.
(60) Provisional application No. 60/782,960, filed on Mar. 15, 2006.

(51) Int. Cl. A61K 31/352; A23L 1/30; A61K 8/49

(52) U.S. Cl. CPC A61K 31/352; A61K 8/498; A23L 1/3002
USPC 514/456

(57) ABSTRACT

Provided herein are compositions enriched in polyhydroxylated polymethoxyflavones useful as dietary supplements, food additives, pharmaceutical compositions, nutraceutical compositions and cosmetic compositions.

### 1. CROSS-REFERENCE

This application claims the benefit of U.S. Provisional Application No. 60/782,960, filed Mar. 15, 2006, the content of which is incorporated herein by reference in its entirety.

### 2. FIELD OF THE INVENTION

Provided herein are compositions comprising at least 15% (w/w) or more hydroxylated polymethoxyflavones (PMFs), processes of preparing hydroxylated PMF-enriched compositions, and methods of using hydroxylated PMF-enriched compositions.

### 3. BACKGROUND OF THE INVENTION

Numerous epidemiological as well as laboratory studies suggest that a diet rich in fruit and vegetables has a preventive effect for a variety of cancers and disease. Citrus flavonoids have been of particular interest because many of these flavonoids exhibit a broad spectrum of biological activity, including anti-inflammatory, anti-carcinogenic, anti-tumor, anti-viral, anti-oxidant, anti-thromobogenic and anti-atherogenic properties. Certain types of flavonoids, in particular polymethoxyflavones (PMFs) including 5,6,7,8,4′-pentamethoxyflavone (tangeretin), 5,6,7,3′,4′-pentamethoxyflavone (sinensetin), 5,6,7,8,3′,4′-hexamethoxyflavone (nobiletin), among other PMFs, have been isolated from citrus plant extracts such as orange peel extracts. See, e.g., WO 01/21137 A1, published Mar. 29, 2001; Manthey and Grohmann (2001) J. Agric. Food Chem. 49:3268-3273. Studies suggest that PMFs such as tangeretin and nobiletin are inhibitors of tumor cell growth and may have anti-inflammatory properties. See, e.g., WO 01/21137 A1, published Mar. 29, 2001; U.S. Pat. No. 6,184,246; Manthy et al. (1999) J. Nat. Prod 62:441-444; Manthey and Guthrie (2002) J. Agric. Food Chem. 50:5837-5843. However, the mechanisms by which PMFs exert anti-inflammatory and anti-cancer effects remain largely unexplained. Moreover, it remains to be seen what structural features are important for conferring beneficial activities that are associated with PMFs.

### 4. SUMMARY OF THE INVENTION

In one aspect, provided herein are compositions comprising hydroxylated polymethoxyflavones (PMFs). In particular, provided herein are compositions that comprise at least 15% (w/w) to 95% (w/w), preferably at least about 20% (w/w) to about 90% (w/w), hydroxylated PMFs. The compositions provided can be prepared from, for example, plant extracts such as an extract from a citrus plant, typically, an orange peel extract.

In certain embodiments, provided herein are plant extract compositions comprising a PMF fraction enriched for hydroxylated PMFs. In some embodiments, plant extract composition comprises a PMF fraction having at least 15% (w/w) to 95% (w/w) hydroxylated PMFs.

In certain embodiments, the composition is a dietary supplement, food additive or nutraceutical. In some embodiments, the composition is a cosmetic composition.

Compositions as provided herein can comprise at least two or more hydroxylated PMFs selected from those listed in Table 1. In certain embodiments, the hydroxylated PMFs of a composition provided herein consists essentially of at least two, at least three, at least four, at least five, at least six, or more of the hydroxylated PMFs listed in Table I.

TABLE 1
Hydroxylated PMFs

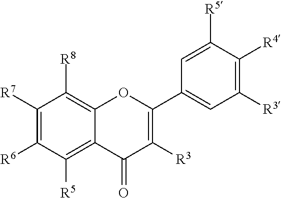

| | |
|---|---|
| 3-hydroxy-5,6,7,4′-tetramethoxyflavone | 5-hydroxy-6,7,4′-trimethoxyflavone |
| 3-hydroxy-5,6,7,8,4′-pentamethoxyflavone | 5,3′-dihydroxy-6,7,8,4′-tetramethoxyflavone |
| 3-hydroxy-5,6,7,8,3′,4′-hexamethoxyflavone | 5-hydroxy-7,8,3′,4′-tetramethoxyflavone |
| 5-hydroxy-3,6,7,3′,4′-pentamethoxyflavone | 5,7-dihydroxy-6,8,3′,4′-tetramethoxyflavone |
| 5-hydroxy-3,6,7,8,3′,4′-hexamethoxyflavone | 7-hydroxy-3,5,6,8,3′,4′-hexamethoxyflavone |
| 5-hydroxy-3,7,8,3′,4′-pentamethoxyflavone | 7-hydroxy-3,5,6,3′,4′-pentamethoxyflavone |
| 5-hydroxy-3,7,3′,4′-tetramethoxyflavone | 3′-hydroxy-5,6,7,4′-tetramethoxyflavone |
| 5-hydroxy-6,7,8,3′,4′-pentamethoxyflavone | 3′-hydroxy-5,6,7,8,4′-pentamethoxyflvone |
| 5-hydroxy-6,7,8,3′,4′,5′-hexamethoxyflavone | 3′,4′-dihydroxy-5,6,7,8-tetramethoxyflavone |
| 5-hydroxy-6,7,8,4′-tetramethoxyflavone | 4′-hydroxy-5,6,7,8,3′-pentamethoxyflavone |
| 5-hydroxy-6,7,3′,4′-tetramethoxyflavone | |

In one aspect, methods are provided for preparing compositions as described herein. In particular, methods for increasing the proportion of hydroxylated PMFs to non-hydroxylated PMFs in a plant extract are provided. In certain embodiments, the methods provided comprise adding acid to a plant extract comprising about 10% (w/w) to about 75% (w/w) PMFs, wherein the non-hydroxylated PMFs are in greater abundance than hydroxylated PMFs; and heating the acidified plant extract to about 40° C. to about 150° C. for about 4 hours to about 36 hours. In certain embodiments, the methods provided comprise adding acid to a plant extract comprising about 20% (w/w) to about 75% (w/w) PMFs, wherein the non-hydroxylated PMFs are in greater abundance than hydroxylated PMFs; and heating the acidified plant extract to about 85° C. to about 100° C. for about 6 hours to about 16 hours. In some embodiments, an acid of about 1 N to about 6 N HCl is added to the plant extract.

As demonstrated herein, hydroxylated PMFs, and compositions thereof, are effective in increasing intracellular calpain and/or intracellular caspase-12 activity in cancer cells leading to apoptosis of the cells. Hence, in one aspect, methods of inhibiting the proliferation of a cancer cell are provided. In certain embodiments, methods provided comprise administering to a mammal, including a human, in need thereof, an amount of a hydroxylated PMF, or composition thereof, that is effective to inhibit proliferation of the cancer cell.

In another aspect, methods of inducing apoptosis in a cancer cell are provided. In some embodiments, methods provided comprise administering to a mammal, such as a human, in need thereof, an amount of a hydroxylated PMF, or composition thereof, that is effective to induce apoptosis of a cancer cell.

In the methods provided, a cancer cell can be, for example, a colon cancer cell, breast cancer cell, leukemia cell or a gastric cancer cell.

In another aspect, methods of inhibiting or reducing inflammation are provided. In certain embodiments, the methods provided comprise administering to a mammal, such as a human, in need thereof, an amount of a hydroxylated PMF, or composition thereof, that is effective to inhibit or reduce inflammation.

In some embodiments, methods are provided for reducing nitrite production in a macrophage comprising contacting the macrophage with a hydroxylated PMF or composition thereof.

In certain embodiments, methods of inhibiting iNOS and/or COX-2 activation in a macrophage are provided, the methods comprising contacting the macrophage with a hydroxylated PMF or composition thereof.

### 5. DESCRIPTION OF THE FIGURES

FIG. 1 provides an exemplary HPLC profile of hydroxylated and non-hydroxylated PMFs separated from a commercially obtained orange peel extract.

FIG. 2 provides experimental results demonstrating the more effective anti-inflammatory properties of hydroxylated PMF-enriched compositions as compared to 5,6,7,8,3′,4′-hexamethoxyflavone (nobiletin) or orange peel extract having a 70% predominantly non-hydroxylated PMF fraction.

FIG. 3 provides data on the effects of PMFs on LPS-induced nitrite production in RAW 264.7 macrophages. *P<0.05, **P<0.01 and ***P<0.001 indicate statistically significant differences from the LPS-treated group.

FIG. 4 provides a comparison of the effects between different PMFs on the expression levels of iNOS, COX-2 and β-actin proteins in macrophages stimulated with LPS.

FIG. 5 provides observed effects of PMFs on LPS-induced NFκB promoter activities in RAW264.7 macrophages using luciferase activity as a reporter. *P<0.05, **P<0.01 and ***P<0.001 indicate statistically significant differences from the LPS-treated group.

FIG. 6 provides experimental results demonstrating that 20 μM 5-hydroxy-3,6,7,8,3′,4′-hexamethoxyflavone inhibits LPS-induced COX-2 mRNA expression in macrophages.

FIG. 7 demonstrates the growth inhibitory effects of PMFs in MCF-7 cells treated with PMFs or vehicle for 1 (A), 3 (B) or 6 (C) days. Results are means of triplicate determinations of two independent experiments.

FIG. 8 demonstrates the proapoptotic effect of PMFs in MCF-7 cells treated with PMFs or vehicle for 1 (A), 3 (B) or 6 (C) days. Results are presented as fluorescence intensity units (FU) per 1×10³ cells.

FIG. 9 demonstrates cell death induced by PMFs in MCF-7 cells treated with PMFs or vehicle for 1 (A), 3 (B) or 6 (C) days. Results are presented as fluorescence intensity units (FU) per 1×10³ cells.

FIG. 10 demonstrates the effects of PMFs on intracellular Ca²⁺ levels in MCF-7 cells treated with PMFs or vehicle for 1 (A), 3 (B) or 6 (C) days.

FIG. 11 demonstrates the effects of PMFs on Ca²⁺ influx and Ca²⁺ mobilization in MCF-7 cells. The Ca²⁺ mobilization responses (A, B) are shown as the maximum [Ca²⁺]ᵢ rises after addition of thapsigargin. The Ca²⁺ entry rates (C, D) are presented as tangents of the linear portions of the fura-2 quench curves. Data in panels A and C are presented as means±SE for control cells or cells treated with PMFs 3 days (black bars) or 6 days (gray bars). Panels B and D show representative traces of the single cell recordings of the Ca²⁺ influx and Ca²⁺ mobilization, respectively, where RFU is relative fluorescence units.

FIG. 12 demonstrates the effects of PMFs on calpain and caspase-12 activity in MCF-7 cells. Calpain (A) and caspase (B) activity were measured with fluorogenic peptide substrates at day 3 (black bars) or day 6 (gray bars) and expressed as percentage of the fluorescently labeled cells (defined as cells with fluorescence intensity at least 2.5-fold above the background cell fluorescence). Data are presented as means±SE; (*), p<0.05, as compared with the corresponding control group.

FIG. 13 demonstrates calpain and caspase-12 activation in PMF-treated MCF-7 cells. Panel A, the cleaved calpain substrate; panel B, the calpain small subunit; panel C, the cleaved caspase-12 substrate; and panel D, the caspase-12 protein. The cells were treated with PMFs for 3 (A, B) or 6 (C, D) days. Upper rows, fluorescence images; lower rows, phase contrast images; a, control; b, compound 4; c, compound 5; d, compound 6; e, compound 7.

FIG. 14 demonstrates the plasma membrane asymmetry and nuclear fragmentation in PMF-treated MCF-7 cells. A, Annexin V-labeled cells; B, Hoesht 33342-labeled cells. The cells were treated with PMFs for 6 (A) or 3 (B) days.

FIG. 15 provides representative HPLC profiles of hydroxylated and non-hydroxylated PMFs separated from starting material fraction (A), hexanes solvent fraction (B), and ethyl acetate solvent fraction (C) of an exemplary method for preparing an enriched hydroxylated PMF composition described in Example 9. Peaks numbered 1-11 correspond to those for PMFs identified in Table 16.

### 6. TERMINOLOGY

Abbreviations used herein include: COX-2, cyclooxygenase-2; G3PDH, glyceraldehyde-3-phosphate dehydrogenase; HPLC, high performance liquid chromatography; LPS, lipopolysaccharide; OPE, orange peel extract; PCR, polymerase chain reaction; and PMF, polymethoxyflavone.

The term “about” as used herein refers to a value that is no more than 10% above or below the value being modified by the term. For example, the term “about 5%” means a range of from 4.5% to 5.5%.

The term “acidified” used herein in context of an “acidified” mixture, where a mixture can be an extract or starting material, etc., means a mixture to which acid has been added, making the mixture more acidic than it was prior to the addition of acid. For example, an acidic mixture can be acidified by the addition of acid to the acidic mixture to make an acidified mixture. For example, a neutral or basic mixture can be acidified by the addition of acid to the neutral or basic mixture to make an acidified mixture. An acidified mixture has a pH lower than the mixture prior to the addition of acid.

As used herein, the term “composition” is meant to encompass dietary supplements, food additives, nutraceuticals, cosmetic compositions, pharmaceutical compositions and physiologically acceptable compositions. It will be understood that where a component, for example, a polymethoxylated flavone (PMF), in a “composition” also occurs in a natural source (for instance, orange peel), the term “composition” does not include the natural source (for instance, orange peel) of the component, but can, in certain embodiments, encompass a physically or chemically modified or processed form of the natural source, such as an extract of the natural source.

The term “effective amount” as used herein refers to the amount of a compound or composition that is sufficient to produce a desirable or beneficial effect when administered, for example, to a subject. In certain embodiments, an “effective amount” of a compound or composition In certain embodiments, an “effective amount” is the amount of a compound or composition sufficient to reduce or ameliorate the severity or duration of a disorder (e.g., a proliferative disorder or an inflammatory disorder) or one or more symptoms thereof, prevent the advancement of a disorder (e.g., a proliferative disorder or an inflammatory disorder), cause regression of a disorder (e.g., a proliferative disorder or an inflammatory disorder), prevent the recurrence, development, or onset of one or more symptoms associated with a disorder (e.g., a proliferative disorder or an inflammatory disorder), or enhance or improve the prophylactic or therapeutic effect(s) of another therapy.

As used herein, the term “isolated” in the context of a compound or composition that can be obtained from a natural source, e.g., plants, refers to a compound or composition that is separated from one or more components from its natural source, preferably, a compound or composition that is substantially free of natural source cellular material, e.g., plant cellular material, or contaminating materials from the natural source, e.g., cell or tissue source, from which it is obtained. The language “substantially free of natural source cellular material” or substantially free of plant cellular material” includes preparations of a compound that has been separated from cellular components of the cells from which it is isolated. Thus, an “isolated” compound or composition is in a form such that its concentration or purity is greater than that in its natural source. For example, in certain embodiments, an “isolated” compound or composition can be obtained by purifying or partially purifying the compound or composition from a natural source. In some embodiments, an “isolated” compound or composition is obtained in vitro in a synthetic, biosynthetic or semisynthetic organic chemical reaction mixture.

As used herein, the terms “manage,” “managing,” and “management” refer to the beneficial effects that a subject derives from a therapy (e.g., a prophylactic or therapeutic agent), if not resulting in a cure of the disease. In certain embodiments, a subject is administered one or more therapies (e.g., one or more prophylactic or therapeutic agents) to “manage” a disease so as to prevent the progression or worsening of the disease.

As used herein, and unless otherwise indicated in context in which it is used, a percentage (“%”) of a composition is intended to mean weight/weight percentage.

The term “polymethoxyflavone” or “PMF” means, unless otherwise indicated, a compound having the formula

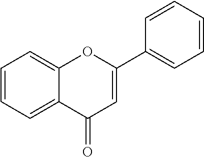 [SMILES: O=c1cc(-c2ccccc2)oc2ccccc12]

wherein at least one carbon, preferably two or more carbons, in the formula are substituted with a —OCH₃ group (in place of one or more hydrogen atoms, not depicted in the formula) as valency permits. As will be clear in the context that the term PMF is used, a PMF may be optionally substituted with substituents, such as, for example, hydroxyl, halide, monosaccharide, or other groups, attached to one or more carbons not substituted with a methoxy group. For example, a “hydroxylated PMF” is a PMF that comprises one or more hydroxyl groups attached to a carbon not substituted with a methoxy group. A “non-hydroxylated PMF” is a PMF that contains no hydroxyl groups.
As used herein, the terms “prevent,” “preventing” and “prevention” refer to the prevention of the recurrence, onset, or development of a disorder or a symptom thereof in a subject resulting from the administration of a compound or composition to the subject.

As used herein, the phrase “prophylactically effective amount” refers to the amount of a therapy (e.g., prophylactic agent) which is sufficient to result in the prevention of the development, recurrence or onset of a disorder or a symptom thereof associated with a disorder (e.g., a proliferative disorder, such as a cancer, or an inflammatory disorder), or to enhance or improve the prophylactic effect(s) of another therapy (e.g., another prophylactic agent).

As used herein, the term “therapeutically effective amount” refers to that amount of a therapy (e.g., a therapeutic agent) sufficient to result in the amelioration of one or more symptoms of a disorder (e.g., a proliferative disorder, such as a cancer, or an inflammatory disorder), prevent advancement of a disorder (e.g., a proliferative disorder or an inflammatory disorder), cause regression of a disorder (e.g., a proliferative disorder or an inflammatory disorder), or to enhance or improve the therapeutic effect(s) of another therapy. In a specific embodiment, with respect to the treatment of cancer, an effective amount refers to the amount of a therapy (e.g., a therapeutic agent) that inhibits or reduces the proliferation of cancerous cells, inhibits or reduces the spread of tumor cells (metastasis), inhibits or reduces the onset, development or progression of cancer or a symptom thereof, or reduces the size of a tumor. Preferably, a therapeutically effective amount of a therapy (e.g., a therapeutic agent) reduces the proliferation of cancerous cells or the size of a tumor by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%, relative to a control or placebo such as phosphate buffered saline (“PBS”). In another embodiment, with respect to inflammation, an effective amount refers to the amount of a therapy (e.g., a therapeutic agent) that reduces the inflammation of a joint, organ or tissue. Preferably, a therapeutically effective amount of a therapy (e.g., a therapeutic agent) reduces the inflammation of a joint, organ or tissue by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%, relative to a control or placebo such as phosphate buffered saline.

As used herein, the terms “therapies” and “therapy” can refer to any protocol(s), method(s), and/or agent(s) that can be used in the prevention, treatment, management, or amelioration of a disorder (e.g., a proliferative disorder or an inflammatory disorder) or one or more symptoms thereof.

As used herein, the terms “treat”, “treatment” and “treating” refer to the reduction or amelioration of the progression, severity and/or duration of a disorder (e.g., a proliferative disorder or an inflammatory disorder), or the amelioration of one or more symptoms thereof resulting from the administration of one or more therapies (e.g., one or more therapeutic agents such as a compound of the invention). In specific embodiments, such terms refer to the inhibition or reduction in the proliferation of cancerous cells, the inhibition or reduction in the spread of tumor cells (metastasis), the inhibition or reduction in the onset, development or progression of cancer or a symptom thereof, the reduction in the size of a tumor, or the improvement in a patient's ECOG or Karnofsky score. In other embodiments, such terms refer to a reduction in the swelling of one or more joints, organs or tissues, or a reduction in the pain associated with an inflammatory disorder. In yet other embodiments, such terms refer to a reduction a human's PASI score or an improvement in a human's global assessment score.

### 7. DETAILED DESCRIPTION

### 7.1. Preparing Hydroxylated Polymethoxyflavone Compositions

In one aspect, provided herein are methods of preparing hydroxylated PMF-enriched plant extract compositions. The term “enriched,” as used herein in connection to a “hydroxylated PMF-enriched” plant extract composition, encompasses a plant extract composition wherein hydroxylated PMFs in the plant extract composition comprise at least 15% to about 95% of the total weight of the plant extract composition, and the proportion of hydroxylated PMFs to non-hydroxylated PMFs in plant extract composition is greater than the proportion hydroxylated PMFs to non-hydroxylated PMFs found naturally in the plant from which the extract is derived. In certain embodiments, a “hydroxylated PMF-enriched” plant extract composition comprises at least 15%, at least about 20%, at least about 25%, at least about 30%, at least about 40%, at least about 45%, at least about 50%, at least about 55%, at least about 60%, at least about 65%, at least about 70%, at least about 75%, at least about 80%, at least about 85%, at least about 90%, or at least about 95% hydroxylated PMFs of the total weight of the composition.

Starting materials for preparing hydroxylated PMF-enriched plant extract compositions typically include an extract or isolate from a natural source that comprises PMFs. In general, sources of PMFs, such as orange peels, for instance, have a PMF fraction in which non-hydroxylated PMFs are more abundant than hydroxylated PMFs. See, e.g., Example 1, below. Hence, in some embodiments, methods are provided for increasing the proportion of hydroxylated PMFs to non-hydroxylated PMFs in a plant extract.

The starting materials for the methods provided herein can be a natural source, typically a plant, plant part, or extract of a plant or plant part, such as an extract of sap, bark, peel, rind, seed, root, juice, leaf, flower, bud, etc., from a plant that naturally contains a measurable PMF component. Citrus products, for example, are a readily available source for obtaining PMFs. In certain embodiments, the plant extract is an orange peel extract, for example, an extract from cold-pressed orange peel oil solids. In some embodiments, compositions for use in the instant methods comprises an extract from Valencia and Hamlin varieties of oranges.

Orange peel extracts that typically comprise between about 20% to about 70% PMFs are commercially available from vendors such as Danisco USA, Inc. (Lakeland, Fla., USA). Typically, an orange peel extract (OPE) is prepared by cold-pressing orange peels to obtain orange peel oil. Orange peel oil usually contains about 0.4% PMFs, a 98% light volatile fraction and 2% residue. A separation process utilizing extraction with solvents followed by drying the extract can be performed to yield an orange peel extract in powder form. Amounts of hydroxylated PMFs contained within commercially available OPEs were determined as described in the Examples below.

In certain embodiments, the starting material for the methods of preparing a hydroxylated PMF-enriched composition is a sweet orange peel extract identified by CAS Registry No. 068917-06-6.

In certain embodiments, the starting material is an orange peel extract having about 10% or about 20% to about 75% PMFs. The PMF fraction can, for example, consist of non-hydroxylated PMFs or comprise non-hydroxylated PMFs and hydroxylated PMFs. The starting material can, for example, be in a liquid form, such as oil or suspension, or in a dried form, such as a powder or paste, and the like.

For example, in certain embodiments, a hydroxylated PMF-enriched composition is prepared from a dried orange peel extract having a PMF fraction of about 10% to about 75% by contacting the orange peel extract with a solvent to form a solution or suspension, adding acid to the solution or suspension to form an acidified mixture, heating the acidified mixture and allowing the mixture to cool, neutralizing the acidified mixture, and extracting the neutralized mixture to obtain the hydroxylated PMF-enriched composition.

In the methods provided, the hydroxylated PMF component of the PMF fraction of the starting material is enriched by adding acid to the starting material and heating the acidified mixture. An acidified mixture is a mixture to which acid has been added.

In some embodiments, the methods comprise adding acid to a starting material comprising about 10% or 20% (w/w) to about 75% (w/w) PMFs, and heating the acidified starting material for an extended period of time, where an extended period of time is longer than one hour, typically longer.

In certain embodiments, the starting material has a PMF fraction having a greater abundance of non-hydroxylated PMFs relative to hydrolyzed PMFs.

In some embodiments, the starting material to be acidified is dissolved or dispersed in a mixture of water and organic solvent. In certain embodiments, the starting material to be acidified is dissolved or dispersed in water. In some embodiments, the starting material is dissolved or dispersed in a organic solvent. Organic solvents are known to those of skill in the art, and are solvents, usually liquid, that contain carbon. Exemplary organic solvents include ethanol, propanol, isopropanol, hexanol, tetrahydrofuran and so forth. In some embodiments, the organic solvent is miscible with water. In certain embodiments, the organic solvent is a polar solvent. Suitable polar solvents include, for instance, water, methanol, ethanol, 1-propanol, isopropanol, 1-butanol, isobutanol, and the like, or a mixture of polar solvents.

Any acid can be added to acidify the starting material, including, for example, a strong acid such as HCl, H₂SO₄, HNO₃, and so forth; an organic acid such as formic acid, trifluoroacetic acid, citric acid, malonic acid, etc.; a weak acid such as acetic acid, phosphoric acid, etc.; or a Lewis acid such as BF₃, BBr₃, BCl₃, and so forth.

In some embodiments, the pH of the acidified starting material can be about 7.0, about 6.5, about 6.0, about 5.5, about 5.0, about 4.5, about 4.0, about 3.5, about 3.0, about 2.5, about 2.0, about 1.5, about 1.0 or about 0.5.

The acidified starting material is heated to enhance the conversion of non-hydroxylated PMFs to hydroxylated PMFs. In certain embodiments, the acidified starting material is heated between about 30° C. to about 250° C. In some embodiments, the acidified starting material is heated between about 40° C. to about 200° C. or between about 50° C. to about 150° C. In certain embodiments, the acidified starting material is heated to about 60° C. to about 100° C. In some embodiments, the acidified starting material is heated to about 60° C., about 65° C., about 70° C., about 75° C., about 80° C., about 85° C., about 90° C., about 95° C., about 100° C., or about 105° C. In some embodiments, the acidified starting material is heated to about 85° C. or to about 100° C. In some embodiments, the acidified starting material is heated between about 40° C. to about 150° C.

The time over which the acidified starting material is heating can, for example, be between about 1 to about 48 hours. In certain embodiments, the acidified starting material is heated between about 5 minutes to about 30 minutes, about 30 minutes to about 1 hour, about 1 hour to about 5 hours, about 5 hours to about 10 hours, about 10 hours to about 15 hours, or about 15 to about 24 hours. In certain embodiments, the time over which the acidified starting material is heated can be about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, or about 19 hours. In some embodiments, the acidified starting material is heated for about 6 hours to about 16 hours. In some embodiments, the acidified starting material is heated for about 4 hours to about 36 hours.

Typically after heating the acidified starting material, the mixture is allowed to cool, usually to room temperature. The mixture can be then neutralized with a base. Any suitable base known to those of skill can be used. Suitable bases include, for example, sodium hydroxide, sodium carbonate, sodium bicarbonate, potassium hydroxide, potassium carbonate, potassium bicarbonate, and the like, or mixtures thereof. The pH of the neutralized mixture can, for example, be between about 5.5 to about 8.5. In certain embodiments, the pH of the neutralized mixture is about 5.8, about 6.0, about 6.5, about 7.0, about 7.5 or about 8.0.

In some embodiments, the methods of preparing hydroxylated PMF-enriched compositions provided herein further comprise extracting the composition produced by heating the acidified starting material. Appropriate solvents for extraction are those that are non-miscible with the solvent within which the product is dissolved or dispersed. For example, in embodiments where ethanol is the solvent used to dissolver or disperse the starting material prior to heating, then solvents such as ethyl acetate can be used for extracting the product after heating. Appropriate solvents will be selected by those of skill in the art.

For example, where ethyl acetate is used to extract a suitably cooled and neutralized product of the acidified starting material in an aqueous solution, the hydroxylated PMFs can be collected in the ethyl acetate fraction.

In other embodiments, where the product of the acidified starting material is in an aqueous solution, a non-polar solvent can be used to extract non-PMFs and/or non-hydroxylated PMFs from the aqueous solution, leaving an enriched hydroxylated PMF fraction in the aqueous fraction. The non-polar solvent can, for example, be a hydrocarbon solvent. Suitable non-polar solvents are those that are non-miscible with the aqueous solution containing the product of the acidified starting material. Exemplary non-polar solvents include, for instance, hexane, pentane, petroleum ethers, heptanes, and the like, or a mixture thereof. In these embodiments, the hydroxylated PMFs remain in the aqueous phase, which itself can be used as a hydroxylated PMF-enriched composition, or can be further processed, for example, by a second extraction, to further purify the hydroxylated PMF fraction.

For example, in certain embodiments where an aqueous solution containing the product of the acidified starting material has been extracted with a non-polar solvent to remove non-PMFs and/or non-hydroxylated PMFs from the aqueous solution, the aqueous solution can be extracted with a second solvent to remove the hydroxylated PMFs from the aqueous solution. By way of example, the product of the acidified starting material, diluted in an aqueous solvent, can be extracted with hexanes, after which the aqueous fraction is extracted with ethyl acetate, and the hydroxylated PMFs can be obtained in the ethyl acetate fraction.

In some embodiments, the methods of preparing hydroxylated PMF-enriched compositions provided herein further comprise drying the composition. For example, the solvents in which the composition is dissolved or dispersed can be removed by evaporation. In certain embodiments, the composition can be dried by lyophilization or freeze-drying.

In some embodiments, a hydroxylated polymethoxyflavone (PMF)-enriched composition as described herein can be prepared by adding one or more isolated hydroxylated PMFs to a natural product, such as, for example, a plant extract.

Exemplary purifications of individual hydroxylated PMFs, such as, for example, those listed in Table 1, from PMF-containing extracts and compositions, are described in the Examples below.

### 7.2. Compositions

In one aspect, provided herein are hydroxylated polymethoxyflavone (PMF)-enriched compositions.

In some embodiments, the composition is a plant extract composition. In certain embodiments, provided are plant extract compositions comprising a polymethoxyflavone (PMF) fraction having between 10% or 15% (w/w) to about 95% (w/w) of one or more hydroxylated PMFs. In certain embodiments, provided are plant extract compositions comprising at least 15% (w/w) to 95% (w/w) hydroxylated PMFs. In some embodiments the plant extract composition comprises about 20% to about 90%, of about 25% to about 85%, of about 40% to about 85%, or of about 50% to about 85% hydroxylated PMFs.

As discussed in Section 7.1 above, the plant from which the plant extract composition is derived can be any plant, or plant part thereof such as sap, bark, peel, rind, seed, root, juice, leaf, flower, bud, etc., that naturally contains a measurable PMF component.

In certain embodiments, the hydroxylated PMF fraction in the compositions provided comprise at least two of the hydroxylated PMFs listed in Table 1. In some embodiments, the hydroxylated PMF fraction comprises at least three, at least four, at least five, at least six, at least seven, at least eight, at least nine, at least ten, at least eleven, at least twelve, at least thirteen, at least fourteen, at least fifteen, at least sixteen, or at least seventeen or more the hydroxylated PMFs selected from the group consisting of the hydroxylated PMFs listed in Table 1.

In certain embodiments, the hydroxylated PMF fraction in the compositions provided consist essentially of two of the hydroxylated PMFs listed in Table 1. In some embodiments, the hydroxylated PMF fraction consists essentially of three, four, five, six, seven, eight, nine, ten, eleven, twelve, thirteen, fourteen, fifteen, sixteen, seventeen or eighteen PMFs selected from the group consisting of the hydroxylated PMFs listed in Table 1.

In certain embodiments, the hydroxylated PMF fraction in the compositions provided comprise at least one, at least two, at least three, or all four of the hydroxylated PMFs selected from the group consisting of 3′-hydroxy-5,6,7,4′-tetramethoxyflavone, 5-hydroxy-3,6,7,8,3′,4′-hexamethoxyflavone, 5,3′-dihydroxy-6,7,8,4′-tetramethoxyflavone and 4′-hydroxy-5,6,7,8,3′-pentamethoxyflavone.

In certain embodiments, the hydroxylated PMF fraction in the compositions provided consist essentially of at least one, at least two, at least three, or all four of the hydroxylated PMFs selected from the group consisting of 3′-hydroxy-5,6,7,4′-tetramethoxyflavone, 5-hydroxy-3,6,7,8,3′,4′-hexamethoxyflavone, 5,3′-dihydroxy-6,7,8,4′-tetramethoxyflavone and 4′-hydroxy-5,6,7,8,3′-pentamethoxyflavone.

In certain embodiments, the compositions provided herein further comprise a member selected from the group consisting of 5-hydroxy-6,7,8,3′,4′-pentamethoxyflavanone, 2′-hydroxy-3,4,4′,5′,6′-pentamethoxychalcone and 2′-hydroxy-3,4,3′,4′,5′,6′-pentamethoxychalcone.

Depending on the manner of use, the compositions of the invention can be, but are not limited to, the form of a dietary supplement, a food additive, a nutraceutical, a cosmetic composition or a pharmaceutical composition.

### 7.2.1. Dietary Supplements, Food Additives and Nutraceuticals

In various embodiments, depending on the intended use and without limitation, a composition of the invention can be in the form of a dietary supplement, a food additive or nutraceutical. Generally, a dietary supplement is consumed by a subject independent of any food composition, unlike a food additive which is incorporated into a food composition during the processing, manufacture, preparation, or delivery of the food composition, or just before its consumption. Accordingly, a food composition of the invention provides, in addition to nutrition, a therapeutic or prophylactic function to the consumer. A “nutraceutical,” as used herein refers to a product prepared, isolated or purified from a food product not usually associated with food, such as an orange peel, for example, intended to be administered to a mammal to have physiological benefit or to prevent or ameliorate a condition or disorder in the mammal, that is, the nutraceutical provides a benefit other than a nutritional benefit, if any.

In various embodiments, the composition of the invention typically comprises one or more consumable fillers or carriers. The term “consumable” means the filler or carrier that is generally suitable for, or is approved by a regulatory agency of the Federal or a state government, for consumption by animals, and more particularly by humans. In certain embodiments, the meaning of the term “dietary supplement” or “food additive” is the meaning of those terms as defined by a regulatory agency of the Federal or a state government, including the United States Food and Drug Administraion.

The dietary supplement, food additive or nutraceutical as provided herein can be used as an anti-inflammatory agent. As such, it can, for example, be used to relieve any adverse health condition that is mediated by NF-κB activation, NF-κB nuclear translocation, and/or binding of NF-κB to DNA, such as but not limited to proliferative disorders and inflammatory disorders. It can be used to relieve any adverse health condition that is mediated by the action of iNOS and/or COX-2 including but not limited to, arthritis, headache, asthma, allergic rash, inflammatory bowel syndrome, joint pain, chronic fatigue, fibromyalgia and the like. The dietary supplement, food additive or nutraceutical can, for example, be used to inhibit macrophage activation, including, for example, nitrite production.

As provided herein, the dietary supplement, food additive or nutraceutical can be used as an anti-cancer agent. For example, it can be used as an anti-oxidant in any condition that involves the action of free radicals. It can, for example, be used to induce apoptosis in a cancer cell. The cancer cell can, for example, be a colon cancer cell, breast cancer cell, leukemia cell, gastric cancer cell. It can, for example, be used to activate intracellular calpain and/or intracellular caspase-12 activity in a cancer cell.

Typically, a dietary supplement, food additive or nutraceutical as provided herein are intended to be orally taken or consumed. The dietary supplement, food additive or nutraceutical can be in a solid form or a liquid form.

For example, a composition as provided herein, such as a dietary supplement, food additive or nutraceutical, can be a reconstitutable powder that, when reconstituted with a liquid, such as drinking water, can provide a beverage. In another embodiment, a composition as provided herein can be incorporated into other foodstuff, such as but not limited to cooking oil, frying oil, salad oil, margarine, mayonnaise or peanut butter. Oils containing the compounds of the invention can be emulsified and used in a variety of water-based foodstuffs, such as drinks. Accordingly, in one embodiment, a food composition can be a beverage, such as but not limited to, fortified mineral water, fortified distilled water, a fruit juice-based beverage, a shake, a milk-based beverage, a dairy product-based beverage, a yoghurt-based beverage, a carbonated water-based beverage, an alcoholic drink, a coffee-based beverage, a green tea-based beverage, a black tea-based beverage, a grain-based beverage, a soybean-based beverage, or a beverage based on plant extracts.

In addition to beverages, the compositions of the present invention may be used as a food additive to be combined with other foodstuff, for example, syrups, starches, grains, or grain flour. Such food composition fortified with the compounds of this invention may be used in the preparation of foodstuffs, such as baked goods, meat products with fillers (e.g., hamburgers, sausages, etc.), cereals, pastas, and soups.

The hydroxylated PMF-enriched compositions can be included in food compositions which also contain a variety of other beneficial components. Non-limiting examples of such optional components are essential fatty acids, vitamins and minerals. These components are well known to those of skill in the art. Additional disclosure describing the contents and production of food compositions comprising such components may be found in e.g., U.S. Pat. Nos. 5,834,048; 5,817,350; 5,792,461; 5,707,657 and 5,656,312, each of which is incorporated herein by reference in their entirety, and the like. Essential fatty acids are involved in cardiovascular health as well as in support of the immune system. An imbalance in these essential fatty acids can lead to poor cholesterol metabolism. Additionally, the immune system function can become impaired, leading to inflammation.

In embodiments where the compositions of the invention are dietary supplements or food additives, vitamins, precursors, and derivatives thereof, minerals, and amino acids can be added to the compositions.

In other embodiments, the compositions of the invention can be added directly to foods so that an effective amount of the compound is ingested during normal meals. Any methods known to those skilled in the art may be used to add to or incorporate the compositions or compounds into natural or processed foodstuff to make the food composition of the invention. Other optional components in a food additive of the invention include but are not limited to anti-caking agent, dessicant, food preservatives, food coloring, artificial sweetner, etc.

### 7.2.2. Cosmetic Compositions

In some embodiments, provided herein are cosmetic compositions comprising hydroxylated PMFs as described above. Also included is a nonexclusive description of various optional and preferred components useful in embodiments of the present invention. As used herein, “safe and effective amount” means an amount of a compound, component, or composition (as applicable) sufficient to significantly induce a positive effect (e.g., confer a noticeable cosmetic benefit), but low enough to avoid serious side effects, (e.g., undue toxicity or allergic reaction), i.e., to provide a reasonable benefit to risk ratio, within the scope of sound medical judgment.

The cosmetic compositions of the present invention are suitable for providing healthful, therapeutic or aesthetic skin benefits by contacting, deposition and/or adhesion to skin and/or hair, or by providing and maintaining body and/or hair hygiene. Suitable cosmetic agents include, but are not limited to those selected from the group consisting of absorbents, anti-acne agents, anti-caking agents, anti-cellulite agents, anti-foaming agents, anti-fungal agents, anti-inflammatory agents, anti-microbial agents, anti-oxidants, antiperspirant/deodorant agents, anti-skin atrophy agents, antiviral agents, anti-wrinkle agents, artificial tanning agents and accelerators, astringents, barrier repair agents, binders, buffering agents, bulking agents, chelating agents, colorants, dyes, enzymes, essential oils, film formers, flavors, fragrances, humectants, hydrocolloids, light diffusers, opacifying agents, optical brighteners, optical modifiers, particulates, perfumes, pH adjusters, sequestering agents, skin conditioners/moisturizers, skin feel modifiers, skin protectants, skin sensates, skin treating agents, skin exfoliating agents, skin lightening agents, skin soothing and/or healing agents, skin detergents, skin thickeners, sunscreen agents, topical anesthetics, vitamins, and combinations thereof.

The cosmetic compositions of the present invention may also comprise a cosmetically-acceptable carrier and any optional components. Suitable carriers are well known in the art and are selected based on the end use application. For example, carriers of the present invention include, but are not limited to, those suitable for application to skin. Preferably, the carriers of the present invention are suitable for application to skin (e.g., sunscreens, creams, milks, lotions, masks, serums, etc.) and nails (e.g., polishes, treatments, etc.). Such carriers are well-known to one of ordinary skill in the art, and can include one or more compatible liquid or solid filler diluents or vehicles which are suitable for application to skin and nails. The exact amount of carrier will depend upon the level of the bonding agent and any other optional ingredients that one of ordinary skill in the art would classify as distinct from the carrier (e.g., other active components). The compositions of the present invention preferably comprise from about 75% to about 99.999%, more preferably from about 85% to about 99.99%, still more preferably from 90% to about 99%, and most preferably, from about 93% to about 98%, by weight of the composition, of a carrier.

The carrier and compositions herein can be formulated in a number of ways, including but not limited to emulsions (in emulsion technology, a composition comprising a “dispersed phase” and a “continuous phase;” the dispersed phase existing as small particles or droplets that are suspended in and surrounded by a continuous phase). For example, suitable emulsions include oil-in-water, water-in-oil, water-in-oil-in-water, oil-in-water-in-oil, and oil-in-water-in-silicone emulsions. Preferred compositions comprise an oil-in-water emulsion.

The cosmetic compositions of the present invention can be formulated into a wide variety of product types, including creams, waxes, pastes, lotions, milks, mousses, gels, oils, tonics, and sprays. Preferred compositions are formulated into lotions, creams, gels, and sprays. These product forms may be used for a number of applications, including, but not limited to, soaps, shampoos, hair, hand and body lotions, cold creams, facial moisturizers, anti-acne preparations, topical analgesics, make-ups/cosmetics including foundations, eyeshadows, lipsticks, and the like. Any additional components required to formulate such products vary with product type and can be routinely chosen by one skilled in the art.

If compositions of the present invention are formulated as an aerosol and applied to the skin as a spray-on product, a propellant is added to the composition. Examples of suitable propellants include chlorofluorinated lower molecular weight hydrocarbons. A more complete disclosure of propellants useful herein can be found in Sagarin, Cosmetics Science and Technology, 2nd Edition, Vol. 2, pp. 443-465 (1972).

The compositions of the present invention may contain a variety of other components such as are conventionally used in a given product type provided that they do not unacceptably alter the benefits of the invention. These optional components should be suitable for application to mammalian skin, that is, when incorporated into the compositions they are suitable for use in contact with human skin without undue toxicity, incompatibility, instability, allergic response, and the like, within the scope of sound medical or formulator's judgment. The CTFA Cosmetic Ingredient Handbook, Second Edition (1992) describes a wide variety of nonlimiting cosmetic and pharmaceutical ingredients commonly used in the skin care industry, which are suitable for use in the compositions of the present invention. Examples of these ingredient classes include: enzymes, surfactants, abrasives, skin exfoliating agents, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents (e.g., resorcinol, sulfur, salicylic acid, erythromycin, zinc, etc.), anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, polymer beads, film formers, fragrances, humectants, opacifying agents, pH adjusters, propellants, reducing agents, sequestrants, skin bleaching agents (or depigmenting, lightening agents) (e.g., hydroquinone, azelaic acid, caffeic acid, kojic acid, ascorbic acid, magnesium ascorbyl phosphate, ascorbyl glucosamine), skin soothing and/or healing agents (e.g., panthenol and derivatives (e.g., ethyl panthenol), aloe vera, pantothenic acid and its derivatives, allantoin, bisabolol, and dipotassium glycyrrhizinate), thickeners, hydrocolloids, particular zeolites, and vitamins and derivatives thereof (e.g. tocopherol, tocopherol acetate, beta carotene, retinoic acid, retinol, retinoids, retinyl palmitate, niacin, niacinamide, and the like).

Further examples of optional components include wetting agents; emollients; moisturizing agents such as glycerol, PEG 400, thiamorpholinone and derivatives thereof, or urea; anti-seborrhoea agents such as S-carboxymethylcysteine, S-benzylcysteamine, the salts and the derivatives thereof; antibiotics such as erythromycin and esters thereof, neomycin, clindamycin and esters thereof, and tetracyclines; antifungal agents such as ketoconazole or 4,5-polymethylene-3-isothiazolidones; agents for promoting the regrowth of the hair, such as minoxidil (2,4-diamino-5-piperidinopyridine 3-oxide) and derivatives thereof, diazoxide (7-chloro-3-methyl-1,2,4-benzothiadiazine 1,1-dioxide) and phenyloin (5,4-diphenylimidazolidine-2,4-dione); non-steroidal anti-inflammatory agents; carotenoids and, in particular, b-carotene; anti-psoriatic agents such as anthraline and derivatives thereof. The cosmetic compositions according to the invention may also contain flavor-enhancing agents, preserving agents such as para-hydroxybenzoic acid esters, stabilizing agents, moisture regulators, pH regulators, osmotic pressure modifiers, emulsifying agents, UV-A and UVB screening agents, and antioxidants such as butylhydroxyanisole or butylhydroxytoluene.

The compositions of the present invention may include carrier components such as are known in the art. Such carriers can include one or more compatible liquid or solid filler diluents or vehicles that are suitable for application to skin and/or hair.

### 7.2.3. Pharmaceutical Compositions

In certain embodiments, provided herein are compositions comprising a hydroxylated PMF fraction, as described above, or more typically, a hydroxylated PMF compound, such as, for example, a hydroxylated PMF selected from those listed in Table 1, wherein the composition is a pharmaceutical composition. Pharmaceutical compositions of the invention comprise a prophylactically or therapeutically effective amount of a composition or compound described herein, and typically one or more pharmaceutically acceptable carriers or excipients.

In this context, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Examples of suitable pharmaceutical carriers are described in Remington: Science and Practice of Pharmacy, 21^{st} ed., Lippincott Williams & Wilkins, Philadelphia Pa. (2005) and Ansel's Pharmaceutical Dosage Forms and Drug Delivery Systems, 8^{th} ed., Lippincott Williams & Wilkins, Philadelphia Pa. (2004).

Typical pharmaceutical compositions comprise one or more excipients. Suitable excipients are well-known to those skilled in the art of pharmacy, and non-limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition depends on a variety of factors well known in the art including, but not limited to, the way in which the dosage form will be administered to a patient and the specific active ingredients in the dosage form. The pharmaceutical composition, if desired, can also contain minor amounts of wetting or emulsifying agents, or pH buffering agents.

This invention further encompasses anhydrous pharmaceutical compositions and dosage forms comprising active ingredients, since water can facilitate the degradation of some compounds.

A pharmaceutical composition of the invention is formulated to be compatible with its intended route of administration. Examples of routes of administration include, but are not limited to, parenteral, e.g., intravenous, intradermal, subcutaneous, oral (e.g., inhalation), intranasal, transdermal (topical), transmucosal, intra-tumoral, intra synovial and rectal administration. In a specific embodiment, the composition is formulated in accordance with routine procedures as a pharmaceutical composition adapted for intravenous, subcutaneous, intramuscular, oral, intranasal or topical administration to human beings. In a preferred embodiment, a pharmaceutical composition is formulated in accordance with routine procedures for subcutaneous administration to human beings. Typically, compositions for intravenous administration are solutions in sterile isotonic aqueous buffer. Where necessary, the composition may also include a solubilizing agent and a local anesthetic such as lignocamne to ease pain at the site of the injection. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

Generally, the ingredients of pharmaceutical compositions as provided herein are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration. Typical dosage forms of the pharmaceutical compositions comprising a hydroxylated PMF compound, or a pharmaceutically acceptable salt, solvate or hydrate thereof lie within the range of from about 1 mg to about 1000 mg per day, given as a single once-a-day dose in the morning but preferably as divided doses throughout the day taken with food.

### 7.2.4. Unit Dosage Forms

In some embodiments, the compositions as provided herein, that is, in any section or subsection of Section 7.1, can be in a unit dosage form. Preferably, a unit dosage form is a nutraceutical or pharmaceutical composition. Unit dosage forms of the invention comprise a prophylactically or therapeutically effective amount of one or more hydroxylated PMFs or compositions thereof, and typically one or more consumable and/or physiologically or pharmaceutically acceptable carriers or excipients, as described above.

In certain embodiments, unit dosage forms comprise an amount of one or more hydroxylated PMFs, or compositions thereof, effective to inhibit iNOS and/or COX-2 activation in a cell, preferably a macrophage.

In some embodiments, unit dosage forms comprise an amount of one or more hydroxylated PMFs, or compositions thereof, effective to inhibit nitrite production in a macrophage.

In certain other embodiments, unit dosage forms comprise an amount of one or more hydroxylated PMFs, or compositions thereof, effective to induce apoptosis in a cancer cell.

In some embodiments, unit dosage forms comprise an amount of one or more hydroxylated PMFs, or compositions thereof, effective to activate calpain and/or capase 12 in a cancer cell.

The invention further encompasses unit forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Different effective amounts may be applicable for different conditions. Unit dosage forms can, for example, take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Such compositions and dosage forms will contain a prophylactically or therapeutically effective amount of a prophylactic or therapeutic agent preferably in purified form, together with a suitable amount of carrier so as to provide the form for proper administration to the patient. The formulation should suit the mode of administration. In a preferred embodiment, the unit dosage forms are sterile and in suitable form for administration to a subject, preferably an animal subject, more preferably a mammalian subject, and most preferably a human subject.

The composition, shape, and type of dosage forms of the invention will typically vary depending on their use. For example, a dosage form used in the acute treatment of inflammation or a related disorder may contain larger amounts of one or more of the active ingredients it comprises than a dosage form used in the chronic treatment of the same disease. Also, the prophylactically and therapeutically effective dosage form may vary among different types of cancer. Similarly, a parenteral dosage form may contain smaller amounts of one or more of the active ingredients it comprises than an oral dosage form used to treat the same disease or disorder. These and other ways in which specific dosage forms encompassed by this invention will vary from one another will be readily apparent to those skilled in the art. See, e.g., Remington: Science and Practice of Pharmacy, 21^{st} ed., Lippincott Williams & Wilkins, Philadelphia Pa. (2005); Ansel's Pharmaceutical Dosage Forms and Drug Delivery Systems, 8^{th} ed., Lippincott Williams & Wilkins, Philadelphia Pa. (2004).

In some embodiments, an article of manufacture is provided that can simplify the administration of one or more hydroxylated PMFs or compositions thereof to a subject. A typical article of manufacture of the invention comprises a unit dosage form of a composition or compound of the invention. In one embodiment, the unit dosage form is a container, preferably a sterile container, containing an effective amount of a composition or compound of the invention and a pharmaceutically acceptable carrier or excipient. The article of manufacture can further comprise a label or printed instructions regarding the use of composition or compound or other informational material that advises the dietitian, physician, technician, consumer, subject, or patient on how to appropriately prevent or treat the disease or disorder in question. In other words, the article of manufacture includes instruction means indicating or suggesting a dosing regimen including, but not limited to, actual doses, monitoring procedures, and other monitoring information. In a specific embodiment, the article of manufacture comprises a container containing an effective amount of a composition or compound of the invention and a pharmaceutically acceptable carrier or excipient. As with any pharmaceutical product and dietary supplement, the packaging material and container included in the article of manufacture are designed to protect the stability of the product during storage and shipment.

Article of manufacture of the invention can further comprise devices that are useful for administering the unit dosage forms. Examples of such devices include, but are not limited to, syringes, drip bags, patches, and inhalers.

Articles of manufacture of the invention can further comprise pharmaceutically acceptable vehicles or consumable vehicles that can be used to administer one or more active ingredients (e.g., a compound of the invention). For example, if an active ingredient is provided in a solid form that must be reconstituted for parenteral or oral/enteral administration, the article of manufacture can comprise a sealed container of a suitable vehicle in which the active ingredient can be dissolved. For parenteral administration, a particulate-free sterile solution is preferred. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### 7.2.5. Oral Dosage Forms

Compositions as provided herein can, for example, be suitable for oral administration, and orally consumable compositions including but not limited to dietary supplements of the invention, can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington: Science and Practice of Pharmacy, 21^{st} ed., Lippincott Williams & Wilkins, Philadelphia Pa. (2005); Ansel's Pharmaceutical Dosage Forms and Drug Delivery Systems, 8^{th} ed., Lippincott Williams & Wilkins, Philadelphia Pa. (2004).

Typical oral dosage forms of the invention are prepared by combining the active ingredient(s) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents. Other ingredients that can be incorporated into the dietary supplement or pharmaceutical compositions of the present invention, may include, but are not limited to, vitamins, amino acids, an antioxidant, a botanical extract, metal salts, and minerals.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical/nutraceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Examples of fillers suitable for use in the pharmaceutical compositions, dietary supplements, and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition, dietary supplement, or dosage form.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof A specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant.

Disintegrants that can be used in pharmaceutical compositions, dietary supplmenents and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and mixtures thereof.

Lubricants that can be used in pharmaceutical compositions, dietary supplements, and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions, dietary supplements, or dosage forms into which they are incorporated.

In certain embodiments, one or more hydroxylated PMFs in a composition as provided herein can be in a delayed release form. For example, the active ingredient can be administered by controlled release means or delivery devices that are well known to those of skill in the art, including, but not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference in its entirety.

### 7.2.6. Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate; and benzyl benzoate. Compounds that increase the solubility of one or more of the active ingredients disclosed herein can also be incorporated into the parenteral dosage forms of the invention.

### 7.2.7. Transdermal, Topical & Mucosal Dosage Forms

Transdermal, topical, and mucosal dosage forms of the invention include, but are not limited to, ophthalmic solutions, sprays, aerosols, creams, lotions, ointments, gels, solutions, emulsions, suspensions, or other forms known to one of skill in the art. See, e.g., Remington: Science and Practice of Pharmacy, 21^{st} ed., Lippincott Williams & Wilkins, Philadelphia Pa. (2005); Ansel's Pharmaceutical Dosage Forms and Drug Delivery Systems, 8^{th} ed., Lippincott Williams & Wilkins, Philadelphia Pa. (2004). Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form lotions, tinctures, creams, emulsions, gels or ointments, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16th and 18th eds., Mack Publishing, Easton Pa. (1980 & 1990).

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

### 7.3. Methods Using Hydroxylated PMFs and Compositions Thereof

In one aspect, provided herein are methods of using the hydroxylated PMFs and compositions thereof as anti-inflammatory agents or anti-proliferation agents. As demonstrated in the Examples below, hydroxylated PMFs are demonstrated to have antiproliferative effects, including, for example, inducing apoptosis in cancer cells. Moreover, hydroxylated PMFs can inhibit inflammation, for example, inhibit iNOS and/or COX-2 production and/or nitrite production in cells such as macrophages involved in an inflammatory response.

Adverse health conditions, diseases and disorders which can be prevented, treated, managed, or ameliorated by administering an effective amount of one or more compounds or compositions of the invention include, but are not limited to, proliferative disorders and inflammatory disorders, and symptoms thereof.

### 7.3.1. Proliferative Disorders

One or more hydroxylated PMF or composition thereof can be used to prevent, treat, manage, or ameliorate a proliferative disorder or one or more symptoms thereof. In certain embodiments, provided herein are methods for preventing, treating, managing, or ameliorating one or more symptoms of a non-cancerous disorder associated with cellular hyperproliferation, particularly of epithelial cells (e.g., as in asthma, COPD, pulmonary fibrosis, bronchial hyperresponsiveness, psoriasis, lymphoproliferative disorder, and seborrheic dermatitis), and endothelial cells (e.g., as in restenosis, hyperproliferative vascular disease, Behcet's Syndrome, atherosclerosis, and macular degeneration), said methods comprising administering to a subject in need thereof one or more hydroxylated PMF or composition thereof.

In a specific embodiment, the invention provides methods for preventing, managing, treating, or ameliorating a non-cancerous disorder associated with cellular hyperproliferation (e.g., Behcet's Syndrome, sarcoidosis, keloids, pulmonary fibrosis, and renal fibrosis) or one or more symptoms thereof, said methods comprising of administering to a subject in need thereof a prophylactically or therapeutically effective amount of one or more hydroxylated PMF or composition thereof.

The present invention provides methods for preventing, treating, managing, or ameliorating cancer or one or more symptoms thereof, said methods comprising administering one or more hydroxylated PMF or composition thereof to a subject in need thereof.

In a specific embodiment, the invention provides a method of preventing, treating, managing, or ameliorating cancer or one or more symptoms thereof, said method comprising administering to a subject in need thereof a dose of a prophylactically or therapeutically effective amount of one or more hydroxylated PMF or compositions thereof.

The compounds of the invention can be used in vitro or ex vivo for the management, treatment or amelioration of certain cancers, including, but not limited to leukemias and lymphomas, such treatment involving, for example, autologous stem cell transplants. This can involve a multi-step process in which the subject's autologous hematopoietic stem cells are harvested and purged of all cancer cells, the patient's remaining hone-marrow cell population is then eradicated via the administration of a high dose of a compound of the invention with or without accompanying high dose radiation therapy, and the stem cell graft is infused back into the subject. Supportive care is then provided while bone marrow function is restored and the subject recovers.

In further embodiments, cancers that can be prevented, managed, treated or ameliorated in accordance with the methods of the invention include, but are not limited to, neoplasms, tumors (malignant and benign) and metastases, or any disease or disorder characterized by uncontrolled cell growth. The cancer may be a primary or metastatic cancer. Specific examples of cancers that can be prevented, managed, treated or ameliorated in accordance with the methods of the invention include, but are not limited to, cancer of the head, neck, eye, mouth, throat, esophagus, chest, bone, lung, colon, rectum, stomach, prostate, breast, ovaries, kidney, liver, pancreas, and brain. Additional cancers include, but are not limited to, the following: leukemias such as but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease, and inflammatory breast cancer; gastric or stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma. For a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America, each of which is incorporated herein by reference in its entirety for all purposes.

In certain embodiments, the methods provided comprise contacting a cancer cell with an amount of hydroxylated PMF or composition thereof effective to induce apoptosis in the cancer cell. In some embodiments, the activated apoptosis is a calcium-mediated apoptosis.

In certain embodiments, the methods provided comprise contacting a cancer cell with hydroxylated PMF or composition thereof in an amount effective to activate calpain and/or caspase-12.

### 7.3.2. Inflammation

One or more hydroxylated PMF or composition thereof can be used to prevent, treat, manage, relieve, or ameliorate an inflammatory disorder or one or more symptoms thereof.

The one or more hydroxylated PMF or composition thereof can be used to prevent, reduce, or eliminate the symptoms and conditions associated with inflammation. A common feature of inflammation is the releases of cytokines or other agents that potently activate inducible cyclo-oxygenase 2 (COX-2) and inducible nitric oxide synthase (iNOS). Gilman A, Rail T, Nies A, Taylor P eds, Goodman and Gilman's The Pharmacological Basis of Therapeutics, New York, Pergamon Press, 1990. Robak J, Gryglewski R J, Bioactivity of flavonoids, Pol J Pharmacol, 1996, 48:555-564. In certain embodiments of the methods provided, COX-2 and/or iNOS expression is inhibited in a cell by contacting the cell with one or more hydroxylated PMF or composition thereof.

In a specific embodiment, the invention provides a method of preventing, treating, managing, or ameliorating a condition associated with inflammation (e.g., an inflammatory disorder) or one or more symptoms thereof, said method comprising contacting with or administering to a subject in need thereof a dose of a prophylactically or therapeutically effective amount one or more hydroxylated PMFs or composition thereof.

Examples of the inflammatory disorders which can be prevented, managed, treated, or ameliorated in accordance with the methods of the invention, include, but are not limited to, asthma, allergic reactions, allergic disorders, inflammatory disorders characterized by type-1 mediated inflammation, inflammatory disorders characterized by type-2 mediated inflammation, fibrotic disease (e.g., pulmonary fibrosis), psoraisis, multiple sclerosis, systemic lupus erythrematosis, chronic obstructive pulmonary disease (COPD), encephilitis, inflammatory bowel disease (e.g., Crohn's disease and ulcerative colitis), ischemic reperfusion injury, Gout, Behcet's disease, septic shock, undifferentiated spondyloarthropathy, undifferentiated arthropathy, arthritis, rheumatoid arthritis (juvenile and adult), osteoarthritis, psoriatic arthritis, inflammatory osteolysis, sepsis, meningitis, and chronic inflammation resulting from chronic viral or bacteria infections.

In a specific embodiment, an amount of one or more hydroxylated PMFs or composition thereof is administered to a subject effective to treat, manage or ameliorate asthma.

### 7.3.3. Dosage & Frequency of Administration

The amount of the one or more hydroxylated PMFs or composition thereof which will be effective in the prevention, treatment, management, relief, or amelioration of an adverse health condition, a disorder (e.g., a proliferative disorder or an inflammatory disorder), or one or more symptoms thereof will vary with the nature and severity of the disease or condition, and the route by which the active ingredient is administered. The frequency and dosage will also vary according to factors specific for each subject or patient depending on the specific therapy (e.g., therapeutic or prophylactic agents) administered, the severity of the disorder, disease, or condition, the route of administration, as well as age, body, weight, response, and the past medical history of the patient. Effective doses may be extrapolated from dose-response curves derived from in vitro or animal model test systems. Suitable regiments can be selected by one skilled in the art by considering such factors and by following, for example, dosages reported in the literature and recommended in the Physician's Desk Reference (57th ed., 2003).

Exemplary doses of a small molecule include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram).

In general, the recommended daily dose range of a one or more hydroxylated PMFs or composition thereof for the conditions described herein lie within the range of from about 0.01 mg of the one or more hydroxylated PMF to about 1000 mg one or more hydroxylated PMF per day. These amounts can, for example, be given as a single once-a-day dose or as divided doses throughout a day. In one embodiment, the daily dose is administered twice daily in equally divided doses. Specifically, a daily dose range should be from about 5 mg to about 500 mg per day, more specifically, between about 10 mg and about 200 mg per day. In managing the subject or patient, the therapy should be initiated at a lower dose, perhaps about 1 mg to about 25 mg, and increased if necessary up to about 200 mg to about 1000 mg per day as either a single dose or divided doses, depending on the subject or patient's global response. It may be necessary to use dosages of the active ingredient outside the ranges disclosed herein in some cases, as will be apparent to those of ordinary skill in the art. Furthermore, it is noted that the dietitian, clinician or treating physician will know how and when to interrupt, adjust, or terminate therapy in conjunction with individual patient responses and conditions, as will be readily known by those of ordinary skill in the art. Similarly, amounts sufficient to prevent, manage, treat or ameliorate such disorders, but insufficient to cause, or sufficient to reduce, adverse effects associated with the compounds of the invention are also encompassed by the above described dosage amounts and dose frequency schedules. Further, when a subject or patient is administered multiple dosages of a compound of the invention, not all of the dosages need be the same. For example, the dosage administered to the subject or patient may be increased to improve the prophylactic or therapeutic effect of the compound or it may be decreased to reduce one or more side effects that a particular subject or patient is experiencing.

In a specific embodiment, the dosage of the one or more hydroxylated PMFs or composition thereof administered to prevent, treat, manage, or ameliorate a disorder (e.g., a proliferative disorder or an inflammatory disorder), or one or more symptoms thereof in a patient is about 150 μg/kg, preferably about 250 μg/kg, about 500 μg/kg, about 1 mg/kg, about 5 mg/kg, about 10 mg/kg, about 25 mg/kg, about 50 mg/kg, about 75 mg/kg, about 100 mg/kg, about 125 mg/kg, about 150 mg/kg, or about 200 mg/kg or more of a patient's body weight. In another embodiment, the dosage of the one or more hydroxylated PMFs or composition thereof administered to prevent, treat, manage, or ameliorate a disorder (e.g., a proliferative disorder or an inflammatory disorder), or one or more symptoms thereof in a patient is a unit dose of 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7 mg, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg.

### 7.4. Biological Assays

Several aspects of the one or more hydroxylated PMFs or compositions thereof can be tested in vitro, in a cell culture system, and in an animal model organism, such as a rodent animal model system, for the desired therapeutic activity prior to use in humans. For example, assays which can be used to determine whether administration of a specific composition is indicated, include cell culture assays in which a patient tissue sample is grown in culture, and exposed to or otherwise contacted with a composition, and the effect of such composition upon the tissue sample is observed. The tissue sample can be obtained by biopsy from the patient. This test allows the identification of the therapeutically most effective therapy (e.g., prophylactic or therapeutic agent(s)) for each individual patient. In various specific embodiments, in vitro assays can be carried out with representative cells of cell types involved in a disorder (e.g., immune cells or cancer cells), to determine if a composition of the invention has a desired effect upon such cell types. As an alternative to the use of tissue, tissue samples, cancer cell lines can be used in in vitro assays. Examples of cancer cell lines that can be utilized in in vitro assays include, but are not limited to, the MCF-7 breast cancer cell line, the MCF-7/ADR multi-drug resistant breast cancer cell line, the HT114 human melanoma cell line, the MES/DOX doxorubicenresistant human uterine sarcoma cell line, the HT29 human colorectal cell line, the HCT-116 human colorectal cell line, the A549 human lung Carcinoma cell line and the BXPC-3 human pancreas primary adenocarcinoma cell line, including cell lines described in the Examples below.

The one or more hydroxylated PMFs or compositions thereof can be assayed for their ability to modulate the activation of various types of immune cells (including T cells, B cells, NK cells, macrophages, and dendritic cells). Activation of immune cells can be determined by measuring, e.g., changes in the level of expression and/or phosphorylation of cytokines, and/or cell surface markers. Techniques known to those of skill in the art, including, but not limited to, immunoprecipitation followed by Western blot analysis, ELISAs, flow cytometry, Northern blot analysis, and RT-PCR can be used to measure the expression of cytokines and cell surface markers indicative of activation of the immune cell.

The one or more hydroxylated PMFs or compositions thereof can be assayed for their ability to induce the expression and/or activation of a gene product (e.g., cellular protein or RNA) and/or to induce signal transduction in immune cells, cancer cells, and/or endothelial cells. The induction of the expression or activation of a gene product or the induction of signal transduction pathways in immune cells, cancer cells (in particular tubulin-binding agent resistant cancer cells) and/or endothelial cells can be assayed by techniques known to those of skill in the art including, e.g., ELISAs, flow cytometry, Northern blot analysis, Western blot analysis, RT-PCR kinase assays and electrophoretic mobility shift assays. The one or more hydroxylated PMFs or compositions thereof can also be assayed for their ability to modulate immune cell proliferation, endothelial and cell cancer cell proliferation. Techniques known to those in art, including, but not limited to, ³H-thymidine incorporation, trypan blue cell counts, and fluorescence activated cell sorting (“FACS”) analysis. The one or more hydroxylated PMFs or compositions thereof can also be assayed for their ability to induce cytolysis. Cytolysis can be assessed by techniques known to those in art, including, but not limited to, ⁵¹Cr-release assays.

The one or more hydroxylated PMFs or compositions thereof can be tested in suitable animal model systems prior to use in humans. Such animal model systems include, but are not limited to, rats, mice, chicken, cows, monkeys, pigs, dogs, rabbits, etc. Any animal system well-known in the art may be used. In a specific embodiment of the invention, the compositions and compounds of the invention are tested in a mouse model system. Such model systems are widely used and well-known to the skilled artisan. Pharmaceutical compositions of the invention can be administered repeatedly. Several aspects of the procedure may vary including, but not limited to, temporal regime for administration of the one or more hydroxylated PMFs or compositions thereof.

The anti-cancer activity of the one or more hydroxylated PMFs or compositions thereof can be determined using any suitable animal model, including, but not limited to, SCID mice with a tumor or injected with malignant cells. Examples of animal models for lung cancer include, but are not limited to, lung cancer animal models described by Zhang & Roth (1994, In Vivo 8(5):755-69) and a transgenic mouse model with disrupted p53 function (see, e.g., Morris et al., 1998, J La State Med Soc 150(4):179-85). An example of an animal model for breast cancer includes, but is not limited to, a transgenic mouse that overexpresses cyclin D1 (see, e.g., Hosokawa et al., 2001, Transgenic Res 10(5):471-8). An example of an animal model for colon cancer includes, but is not limited to, a TCR b and p53 double knockout mouse (see, e.g., Kado et al., 2001, Cancer Res 61(6):2395-8). Examples of animal models for colorectal carcinomas include, but are not limited to, Apc mouse models (see, e.g., Fodde & Smits, 2001, Trends Mol Med 7(8):369-73 and Kuraguchi et al., 2000, Oncogene 19(50):5755-63).

The anti-inflammatory activity of the one or more hydroxylated PMFs or compositions thereof can be determined by using various experimental animal models of inflammatory arthritis known in the art and described in Crofford L. J. and Wilder R. L., “Arthritis and Autoimmunity in Animals”, in Arthritis and Allied Conditions: A Textbook of Rheumatology, McCarty et al. (eds.), Chapter 30 (Lee and Febiger, 1993).

Animal models for asthma can also be used to assess the efficacy of the compositions and compounds of the invention. An example of one such model is the murine adoptive transfer model in which aeroallergen provocation of TH 1 or TH2 recipient mice results in TH effector cell migration to the airways and is associated with an intense neutrophilic (TH1) and eosinophilic (TH2) lung mucosal inflammatory response (Cohn et al., 1997, J. Exp. Med. 1861737-1747).

Further, any assays known to those skilled in the art can be used to evaluate the prophylactic and/or therapeutic utility of the one or more hydroxylated PMFs or compositions thereof for the disorders disclosed herein.

The toxicity and/or efficacy of the one or more hydroxylated PMFs or compositions thereof can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Hyrdroxylated PMFs that exhibit large therapeutic indices are preferred.

The data obtained from the cell culture assays and animal studies can be used in formulating a range of dosage of the compositions and compounds of the invention for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography (HPLC) and radioimmunasssay (RIA). The pharmacokinetics of a prophylactic or therapeutic can be determined, e.g., by measuring parameters such as peak plasma level (Cₘₐₓ), area under the curve (AUC, which is measured by plotting plasma concentration of the agent versus time, and reflects bioavailability), half-life of the compound (t_{1/2}), and time at maximum concentration.

Efficacy in preventing or treating a proliferative disorder such as cancer may be demonstrated, e.g., by detecting the ability of the compositions and compounds of the invention to reduce one or more symptoms of the proliferative disorder, to reduce the proliferation of cancerous cells, to reduce the spread of cancerous cells, or to reduce the size of a tumor. Efficacy in preventing or treating an inflammatory disorder may be demonstrated, e.g., by detecting the ability of the compositions and compounds of the invention to reduce one or more symptoms of the inflammatory disorder, to decrease T cell activation, to decrease T cell proliferation, to modulate one or more cytokine profiles, to reduce cytokine production, to reduce inflammation of a joint, organ or tissue or to improve quality of life. Changes in inflammatory disease activity may be assessed through tender and swollen joint counts, patient and physician global scores for pain and disease activity, and the ESR/CRP. Progression of structural joint damage may be assessed by quantitative scoring of X-rays of hands, wrists, and feet (Sharp method). Changes in functional status in humans with inflammatory disorders may be evaluated using the Health Assessment Questionnaire (HAQ), and quality of life changes are assessed with the SF-36.

### 8. EXAMPLES

The following examples are intended only to further illustrate the invention and are not intended to limit the scope of the invention as defined by the claims.

### 8.1. Example 1

This example presents an analysis of the hydroxylated PMF content of commercially available orange peel extracts.

Orange peel extracts were commercially obtained and determined to have a PMF fraction as follows: Extract A, 70% PMF fraction; Extract B, 40% PMF fraction; Extract C, 40% PMF fraction; Extract D, 20% PMF fraction.

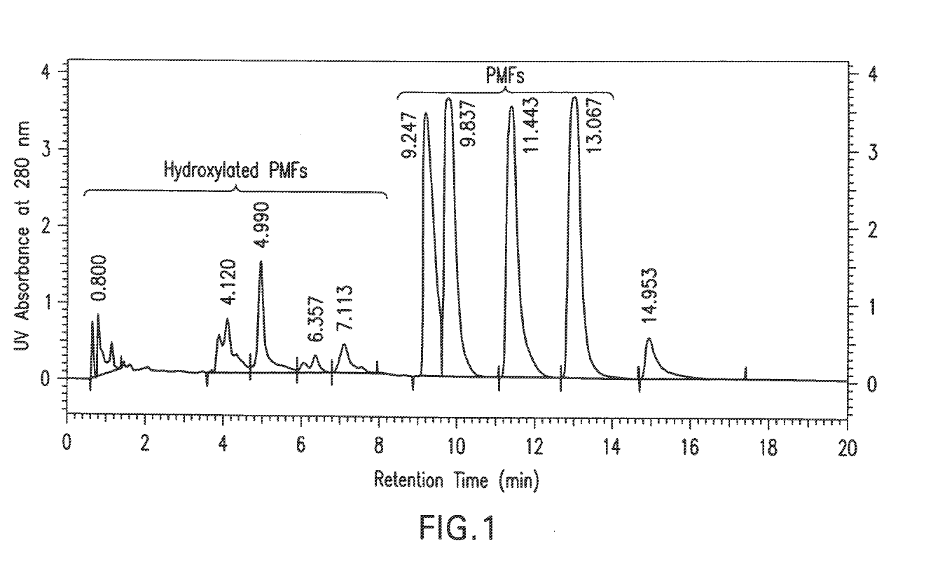

High performance liquid chromatography (HPLC) was utilized to separate hydroxylated PMFs from non-hydroxylated PMFs in orange peel extracts. For each of the four orange peel extracts, a sample was dissolved in methylene chloride in a concentration of 2 mg/ml. The dissolved solutions were analyzed on an HPLC system (Shimadzu Scientific Instruments, Columbia, Md., USA) with vendor-provided auto injector (SIL-10 AD vp), UV-Vis detector (SPD-10A vp), dual pumps (LC/-10 AT vp) and system controller (SCL-10A vp) components with a NOVA-PAK° silica (3.9×150 mm, 5 μm) analytical column (Waters Corp., Milford, Mass., USA) using a gradient mobile phase of 15% ethyl acetate and 85% hexanes to 50% ethyl acetate and 50% hexanes in 15 min runs with a flow rate of 2 mL/min. The monitoring UV absorbance was set at 280 nm. FIG. 1 depicts an exemplary HPLC separation profile from a commercially available orange peel extract where peaks corresponding to hydroxylated PMFs and non-hydroxylated PMFs are as indicated. HPLC peak fractions were characterized as hydroxylated PMFs or non-hydroxylated PMFs by collecting and concentrating the peak fractions and analyzing the concentrated samples by HPLC-electron spray ionization-mass spectrometry (HPLC-ESI-MS).

Briefly, HPLC-ESI-MS was performed on an HP1090 system controller, with a variable UV wavelength 190-500 nm detector, an evaporizing laser scattered deposition detector and an ESI-MS detector from a VG PLATFORM II mass analyzer (Micromass, Beverly, Mass., USA). ESI-MS conditions were as follows: acquisition mode, ESI-positive; mass scan range, 100-800 amu; scan rate, 0.4 sec; cone voltage, 25 volts; source temperature: 150° C.; probe temperature: 550° C. Analytical HPLC conditions on HPLC-MS: column: CHROMABOND WR C18, 3 μm, 120 Å; length×O.D.: 30×3.2 mm; injection volume, 15 μL; flow rate: 2 mL/min; run time: 3 min. Mobile phase consisted of acetonitrile and H₂O with 0.05% TFA, using a typical gradient of 10-90% acetonitrile.

Table 2 provides percentages (w/w) of the fraction indicated to the orange peel extract for each of the four extracts analyzed.

TABLE 2
Concentrations of Hydroxylated PMFs in
Commercially Available OPEs

| | Combined hydroxylated and | |
|---|---|---|
| | non-hydroxylated PMFs, | Hydroxylated PMFs, |
| | % (w/w) | % (w/w) |
| Extract A | 70 | 13.14 |
| Extract B | 40 | 14.75 |
| Extract C | 40 | 9.65 |
| Extract D | 20 | 9.19 |

### 8.2. Example 2

This example describes the characterization of exemplary hydroxlyated compounds isolated from a commercially available orange peel extract. In particular, eight hydroxylated flavones, one hydroxylated flavanone and two hyroxylated chalcones were isolated from a sweet orange peel extract and characterized.

Sweet orange peel extract was obtained from Florida Flavors Co. Thin Layer Chromatography (TLC) plates and pre-packed silica gel (60 Å, 32-63 μm) columns, whose size are 4 g, 12 g, 40 g, 80 g, 120 g and 330 g, for normal phase chromatography and octadecyl (C₁₈) derivatized silica gel (60 Å) for reversed phase flash chromatography were purchased from Teledyne Isco, Inc. (Lincoln, Nebr., USA).

Flash Column System.

An automated flash chromatography system (Model Foxy 200, sg100, ISCO Inc., Lincoln, Nebr., USA) equipped with a 330 g prepacked silica gel (particle size 35 to 60 μm) flash column from Teledyne Isco Inc. was used. The mobile phase for normal phase flash column consisted of either ethyl acetate and hexanes or isopropanol and hexanes in varying proportions and the flow rate was set at 90 mL/min. The eluent was monitored with a single channel UV detector at a wavelength of 254 nm.

HPLC System.

A high performance liquid chromatograph (HPLC) equipped with a pump (Waters Delta Prep 4000 delivery pump, Milford, Mass.), UV-vis detector (Waters 486 tunable absorbance detector, Milford, Mass.) and an injector (Waters U6K injector, Milford, Mass.) was used. A Regis Welk-O 1 R,R 450 gram column (Regis Technologies, Inc., Morton Grove, Ill.) was used for the HPLC system. The mobile phase for the HPLC system was 35% absolute ethanol and 65% hexanes with a flow rate set at 85 mL/min. The eluent was detected with a UV wavelength at 326 nm.

NMR Instrument.

NMR spectra were recorded on a Varian 300 and Varian 500 Spectrometer (Varian Inc., Palo Alto, Calif.). With TMS serving as internal standard, ¹H NMR was recorded at 300 MHz and 500 MHz; ¹³C NMR at 75 MHz and 125 MHz; 2D NMR(HMBC, HSQC, HMQC and ROESY) at 125 MHz.

Mass Spectrometer.

ESI-MS spectra were obtained on a Micromass VG Platform II mass analyzer (Micromass, Beverly, Mass.). ESI-MS spectra were obtained on a MicroMass AutoSpec HF (Micromass, Beverly, Mass.). MS conditions: mass scan range: 100-1500 amu; scan rate: 0.4 sec (ESI-MS); cone voltage: 36 volts (ESI-MS); corona voltage: 3.59 K Volts (ESI-MS); source temperature: 150° C. (ESI-MS); 250° C. (EI-MS).

Additional analytical methods, including HPLC-ESI-MS, were performed similar to that described in Section 8.1, above.

To separate components in orange peel extract, the extract (10 g) was dissolved in a mixture of methylene chloride (2 mL) and hexanes (2 mL) and loaded onto the preconditioned silica gel flash column (size: 330 g). The gradient was started with 10% ethyl acetate and 90% hexanes and went to 40% ethyl acetate and 60% hexanes within 35 min. Then the isocratic mobile phase was applied for 15 min. The fractions that had UV absorbance at 254 nm were analyzed by LC/MS and on thin layer chromatography (TLC) with a solvent system of 40% ethyl acetate and 60% hexanes. The fractions were combined into several groups according to their molecular weight obtained from LC/MS analysis. Further separation of each group was done.

The fractions that contain hydroxylated PMFs characterized by LC/MS were concentrated and the residue was dissolved in acetonitrile and water. The dissolved solution was loaded onto a C18 reverse phase HPLC system. A gradient method was used from 25% acetonitrile to 60% acetonitrile in 25 min. The fractions were analyzed by LC/MS. Both the pure compounds and mixtures were collected. The pure fractions by HPLC and MS were combined and concentrated or lyophilized to remove acetonitrile. The compounds were analyzed by MS and NMR. The mixtures were concentrated on rotovapor and dissolved in minimum amount of methylene chloride. The solution was loaded onto the HPLC system equipped with the Regis column (Welk-O 1R,R 450 gram). The monitoring UV absorbance was set at 280 nm. The fractions were collected and concentrated respectively. MS and NMR were taken for these fractions.

Table 3 depicts the hydroxylated PMFs isolated from the orange peel extract.

TABLE 3
Hydroxylated Polymethoxyflavones Isolated from Sweet Orange Peel Extract

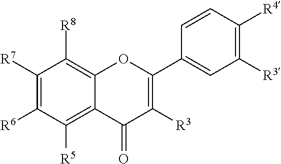

| PMF | R³ | R⁵ | R⁶ | R⁷ | R⁸ | R³′ | R⁴′ |
|---|---|---|---|---|---|---|---|
| 5-hydroxy-6,7,4′-trimethoxyflavone | H | OH | OMe | OMe | H | H | OMe |
| 5-hydroxy-6,7,8,4′-tetramethoxyflavone | H | OH | OMe | OMe | OMe | H | OMe |
| 3-hydroxy-5,6,7,4′-tetramethoxyflavone | OH | OMe | OMe | OMe | H | H | OMe |
| 3-hydroxy-5,6,7,8,4′-pentamethoxyflavone | OH | OMe | OMe | OMe | OMe | H | OMe |
| 5-hydroxy-3,6,7,8,3′,4′-hexamethoxyflavone | OMe | OH | OMe | OMe | OMe | OMe | OMe |
| 5-hydroxy-3,7,3′,4′-tetramethoxyflavone | OMe | OH | H | OMe | H | OMe | OMe |
| 5-hydroxy-3,7,8,3′,4′-pentamethoxyflavone | OMe | OH | H | OMe | OMe | OMe | OMe |
| 5'-hydroxy 6,7,8,3′,4′ pentamethoxyflavone | H | OH | OMe | OMe | OMe | OMe | OMe |

In addition to hydroxylated polymethoxyflavones, the following three compounds were isolated and characterized from orange peel extract:

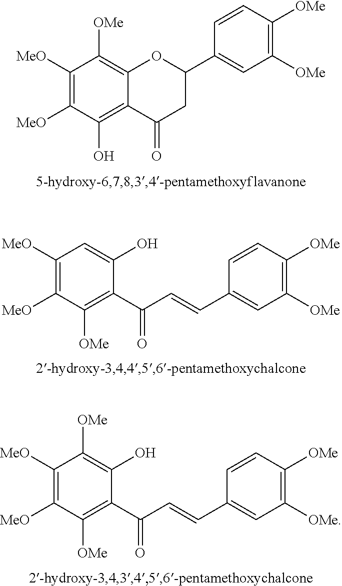 [SMILES: COc1ccc(C2CC(=O)c3c(O)c(OC)c(OC)c(OC)c3O2)cc1OC ; COc1ccc(/C=C/C(=O)c2c(O)cc(OC)c(OC)c2OC)cc1OC ; COc1ccc(/C=C/C(=O)c2c(O)c(OC)c(OC)c(OC)c2OC)cc1OC]

### 8.3. Example 3

This example provides exemplary processes for preparing compositions from orange peel extract to contain greater than 15% (w/w) hydroxylated PMFs.

Treatment with 1 N HCL.

One gram of orange peel extract having 70% PMFs was dissolved in 5 mL of ethanol. Next, 5 mL of 1 N HCl was added to the solution, which was then refluxed at 100° C. for 12 hours. The reaction mixture was extracted twice with 30 mL ethyl acetate. Solvent was removed with rotary evaporator to obtain a dry composition termed “1 N PMF.”

Upon analysis, it was found that the 1 N PMF composition contained about 25.78% hydroxylated PMFs. It was estimated that approximately 31% of nobiletin in the starting orange peel extract was converted to 5-hydroxy-6,7,8,3′,4′-pentamethoxyflavone (5-demethylnobiletin) during the process to prepare the 1 N PMF composition.

Treatment with 6 N HCL.

One gram of orange peel extract having 70% PMFs was dissolved in 5 mL of ethanol. Next, 5 mL of 6 N HCl was added to the solution, which was then refluxed at 100° C. for 12 hours. The reaction mixture was extracted twice with 30 mL ethyl acetate. Solvent was removed with rotary evaporator to obtain a dry composition termed “6 N PMF.”

Upon analysis, it was found that the 6 N PMF composition contained about 84.42% hydroxylated PMFs. It was estimated that approximately 81% of nobiletin in the starting orange peel extract was converted to 5-hydroxy-6,7,8,3′,4′-pentamethoxyflavone (5-demethylnobiletin) during the process to prepare the 6 N PMF composition.

### 8.4. Example 4

This example describes materials and methods, including assays for identifying biological activities of compounds and compositions.

Cell Cultures.

Human colon carcinoma COLO205 and HT-29 cell lines (American Type Culture Collection (ATTC), Manassas, Va.) and the leukemia cell line HL-60 were maintained at 37° C. in 5% CO₂ air in RPMI 1640 medium supplemented with 10% heat-inactivated fetal bovine serum (Gibco BRL, Grand Island, N.Y.), 100 units/mL penicillin, 100 μg/mL streptomycin and 2 mM 1-glutamine. The human gastric carcinoma AGS cell line (CCRC 60102), obtained from the Food Industry Research and Development Institute (Hsinchu, Taiwan), was maintained as described above, except that DMEM/F 12 was used as the medium rather than the RPMI 1640 medium. The human breast carcinoma MCF-7 cell line MCF-7 (ATTC) was cultured in RPMI 1640 medium supplemented with 5% fetal calf serum at 37° C. in a humidified atmosphere of 5% CO₂ in air. The mouse macrophage cell line RAW 264.7 was maintained in RPMI 1640 supplemented with 100 U/ml penicillin, 100 μg/ml streptomycin, and 10% heat-inactivated fetal calf serum.

Cell Survival Assay.

Cancer cells (2×10⁵) were plated in 12-well Petri dishes. The next day, the medium was changed and treated with different concentration of the test compounds or compositions for 24 h. Where individual compounds were to be tested, stock solutions of the compound was prepared to contain 200 μM of the individual hydroxylated or non-hydroxylated PMF in dimethylsulfoxide (DMSO). Control cells were treated with DMSO to a final concentration of 0.05% (v/v). Depending on the objective of the particular experiment, cells were treating with test compound or composition for 1, 3 or 6 days. At the end of incubation, cells were harvested for cell count using a hemocytometer and measuring the cellular total nucleic acid content with the CyQUANT Cell Proliferation Assay Kit (Molecular Probes).

Determination of Apoptotic Ratio (%) and Cell Cycle Distribution.

The human cancer cells (2×10⁵) were cultured in 60-mm Petri dishes and incubated for 24 h. After treated with test compounds for 24 h, the cells were then harvested, washed with PBS resuspended in 200 μL of PBS, and fixed in 800 μL of iced 100% ethanol at −20° C. After being left to stand overnight, the cell pellets were collected by centrifugation, resuspended in 1 mL of hypotonic buffer (0.5% Triton X-100 in PBS and 0.5 μg/mL RNase) and incubated at 37° C. for 30 min. Next, 1 mL of propidium iodide solution (50 μg/mL) was added, and the mixture was allowed to stand on ice for 30 min. Fluorescence emitted from the propidium iodide-DNA complex was quantitated after excitation of the fluorescent dye by FACScan cytometry (Becton Dickinson, San Jose, Calif.).

Nitrite Assay.

Compounds or compositions were tested for anti-inflammatory effects by monitoring nitrite production in LPS-activated RAW264.7 cells. After treating the cells with LPS or LPS and test substance, supernatants were harvested and the amount of nitrite, an indicator of NO synthesis, was measured by use of the Griess reaction. Briefly, supernatants (100 μl) were mixed with the same volume of Griess reagent (1% sulphanilamide in 5% phosphoric acid and 0.1% naphthylethylenediamine dihydrochloride in water) in duplicate on 96-well plates. After incubation at room temperature for 10 min, absorbance at 570 nm was measured with a ELISA reader (Thermo Labsystems Multiskan Ascent, Finland).

Western Blotting.

Proteins were isolated from cells after treatment with test compounds for 24 h. Total protein was extracted by adding 200 μL of gold lysis buffer (50 mM Tris-HCl, pH 7.4; 1 mM NaF; 150 mM NaCl; 1 mM EGTA; 1 mM phenylmethanesulfonyl fluoride; 1% NP-40; and 10 μg/mL leupeptin) to the cell pellets, which were kept on ice for 30 minutes, followed by centrifugation at 10,000×g for 30 min at 4° C. The protein concentrations of the cytosolic fraction (supernatant) were measured by BIO-RAD Protein Assay (Bio-Rad Laboratories, Munich, Germany). Samples containing 50 μg of protein were electrophoretically separated by SDS-PAGE and transferred to PVDF membrane (Millipore Corp., Bedford, Mass.) using standard procedures. See Burnette (1981) Anal. Biochem. 112:195-203. Transferred proteins were visualized by chemiluminesence with reagents from the ECL detection kit (Amersham Pharmacia Biotech, Buckinghamshire, UK) following the manufacturer's protocol using primary anti-iNOS, anti-β-actin and anti-COX-2 antibodies from Transduction Laboratories (Lexington, Ky.) and horseradish peroxide (HRP)-conjugated secondary antibody from Zymed Laboratories (San Francisco, Calif.).

Plasmids, Transient Transfection, and Luciferase Assay.

RAW264.7 cells were seeded in 6 mm dishes. When the cells were confluent, the medium was replaced with serum-free Opti-MEM (Gibco-BRL). Then the cells were transfected with the p-NFκB-Luc plasmid reporter gene using LIPOFECTAMINE™ reagent (Invitrogen). After 6 h incubation, the medium was replaced with complete medium. After 24 h, the cells were trypsinized and equal numbers of cells were plated in 24 well tissue culture plates for 6 h. Cells were then treated with LPS (100 ng/ml) alone or with PMFs (10 or 30 μM) for 12 h. Each well was wash twice with cold PBS and harvested in 100 μl serum-free DMEM, and the luciferase activity was assayed by LUCILITE luciferase reporter gene assay kit (PerkinElamer). Luminescence was measured in a Luminescence counter (Molecular Devices, LMaxII) in single photon counting mode for 2 sec/well, following 5 min adaptation in the dark.

Statistical Analysis.

Statistical significance of differences between control and treated samples were calculated by Student's t-test (Sigmaplot 8.0). p<0.05 was considered significant. Unless otherwise mentioned, all the data shown in the study for cell growth inhibition, quantitative apoptosis, cell cycle phase distribution, nitrite inhibition, and luciferase activity inhibition are representative of 2-3 independent studies.

### 8.5. Example 5

This example demonstrates the increased antiproliferative and apoptosis-inducing activities of hydroxylated PMF-enriched orange peel extracts relative to nobiletin or to orange peel extracts having 70% predominently non-hydroxylated PMFs (“70% PMF OPE”).

Cancer Cell Antiproliferative and Apoptosis-Inducing Activities.

1 N PMF and 6 N PMF compositions containing hydroxylated PMFs, prepared as described in Section 8.3 above, 70% PMF OPE and 5,6,7,8,3′,4′-hexamethoxyflavone (nobiletin) were tested for antiproliferative activity and apoptosis-inducing activity in HL-60, AGS and COLO205 cells as described in Section 8.4 above. Antiproliferative and apoptosis-inducing activities were derived as IC₅₀ values and AC₅₀, values, respectively, where AC₅₀ is the concentration required for 50% apoptosis.

The results, shown in Table 4, indicate that both the hydroxylated PMF-enriched 1 N PMF and 6 N PMF compositions have more potent antiproliferative activities than nobiletin or 70% PMF OPE.

TABLE 4
Antiproliferative (IC₅₀) and apoptosis-inducing (AC₅₀) activities of hydrolyzed
PMF-enriched compositions in the HL-60, AGS, and COLO205 cells

| | HL-60 | | AGS | | COLO205 | |
|---|---|---|---|---|---|---|
| | IC₅₀ μg/ml | AC₅₀ μg/ml | IC₅₀ μg/ml | AC₅₀ μg/ml | IC₅₀ μg/ml | AC₅₀ μg/ml |
| Nobiletin | >100 | >100 | >100 | >100 | 66.73 ± 0.73 | >100 |
| 1N PMF | 41.39 ± 0.07 | >100 | 45.06 ± 0.22 | 75.10 ± 0.24 | 33.60 ± 1.61 | 82.41 ± 7.52 |
| 6N PMF | 23.73 ± 2.18 | >100 | 22.60 ± 1.68 | 93.03 ± 4.04 | 32.56 ± 2.57 | >100 |
| OPE | 43.91 ± 0.31 | >100 | 51.13 ± 0.95 | 88.19 ± 0.40 | 51.68 ± 9.43 | >100 |

Cell Cycle Distribution.

The effects of the hydrolyzed PMF-enriched compositions, nobiletin and 70% PMF OPE on cell cycle distribution were studied, following the procedures described in Section 8.4. Results for cell cycle distribution in HL-60 cells, COLO205 cells and AGS cells are provided in Table 5, Table 6 and Table 7, respectively. Treatment of HL-60, COLO205, and AGS cells for 24 hours resulted in growth arrest involving multiple cell cycle phases. In HL-60 cells, G2/M arrest predominated in cells treated with 6N PMF concentrations ≧5 μM and with 1N PMF concentrations ≧25 μM. In COLO205 cells, G2/M arrest predominated with 1N PMF concentrations ≧25 μM, whereas arrest in G0/G1 was observed with OPE concentrations ≧25 μM. At high 1N PMF concentration (≧25 μM) G2/M and S arrested predominated, whereas at lower 6N PMF concentrations (≧5 μM), G2/M was observed in AGS cells.

TABLE 5
HL-60 Cell Cycle Distributions

| | Concen- | | | |
|---|---|---|---|---|
| | tration | Cell Cycle Distribution | | |
| | (μM) | G0/G1 | S | G2/M |
| Control | — | 51.90 ± 0.13 | 47.76 ± 1.00 | 1.34 ± 1.15 |
| Nobiletin | 5 | 45.81 ± 0.16 | 54.29 ± 0.01** | 0.02 ± 0.01 |
| Nobiletin | 10 | 53.84 ± 0.45 | 40.44 ± 1.06 | 5.73 ± 1.50 |
| Nobiletin | 25 | 61.96 ± 3.55 | 32.12 ± 4.04 | 5.93 ± 0.49 |
| Nobiletin | 50 | 62.37 ± 1.85 | 31.20 ± 1.76 | 6.44 ± 0.08* |
| Nobiletin | 100 | 65.00 ± 3.41* | 30.81 ± 3.08 | 4.18 ± 0.33 |
| 1N PMF | 5 | 48.77 ± 0.13 | 48.01 ± 2.51 | 3.23 ± 0.33 |
| 1N PMF | 10 | 52.24 ± 0.71 | 43.83 ± 0.43 | 3.94 ± 1.14 |
| 1N PMF | 25 | 17.31 ± 4.43 | 47.72 ± 5.39 | 34.98 ± 0.95*** |
| 1N PMF | 50 | 45.71 ± 11.40 | 18.77 ± 26.54 | 35.52 ± 15.15 |
| 1N PMF | 100 | 55.02 ± 1.04 | 0.00 ± 0.00 | 44.99 ± 1.04*** |
| 6N PMF | 5 | 47.71 ± 8.03 | 22.74 ± 28.30 | 29.56 ± 20.27 |
| 6N PMF | 10 | 13.22 ± 1.95 | 43.81 ± 1.82 | 42.98 ± 3.77 |
| 6N PMF | 25 | 12.49 ± 2.53 | 47.52 ± 3.58 | 39.99 ± 6.11 |
| 6N PMF | 50 | 44.27 ± 7.12 | 12.62 ± 17.84 | 43.12 ± 10.72 |
| 6N PMF | 100 | 52.50 ± 1.67 | 0.00 ± 0.00 | 47.50 ± 1.67 |
| OPE | 5 | 45.82 ± 6.82 | 57.00 ± 1.77* | 2.20 ± 2.03 |
| OPE | 10 | 49.83 ± 1.92 | 46.29 ± 4.17 | 3.89 ± 2.26 |
| OPE | 25 | 44.37 ± 2.52 | 0.39 ± 0.54 | 55.26 ± 3.06** |
| OPE | 50 | 36.64 ± 25.31 | 38.73 ± 56.19 | 23.64 ± 30.88 |
| OPE | 100 | 41.65 ± 0.06 | 45.02 ± 0.23 | 13.34 ± 0.29* |

*P < 0.05,
**P < 0.01,
***P < 0.001

TABLE 6
COLO205 Cell Cycle Distributions

| | Concen- | | | |
|---|---|---|---|---|
| | tration | Cell Cycle Distribution | | |
| | (μM) | G0/G1 | S | G2/M |
| Control | — | 57.97 ± 0.24 | 33.78 ± 1.13 | 8.26 ± 1.37 |
| Nobiletin | 5 | 63.83 ± 0.29** | 27.93 ± 0.98 | 8.25 ± 0.69 |
| Nobiletin | 10 | 71.13 ± 0.65** | 21.45 ± 0.17 | 7.42 ± 0.48 |
| Nobiletin | 25 | 84.21 ± 4.38* | 14.29 ± 0.22 | 4.50 ± 0.35 |
| Nobiletin | 50 | 76.82 ± 1.48** | 16.89 ± 1.39 | 6.30 ± 0.08 |
| Nobiletin | 100 | 77.12 ± 2.00** | 17.74 ± 2.40 | 5.11 ± .46 |
| 1N PMF | 5 | 59.44 ± 1.82 | 31.28 ± 2.26 | 9.29 ± 0.44 |
| 1N PMF | 10 | 58.41 ± .21 | 33.21 ± 0.83 | 8.39 ± 1.04 |
| 1N PMF | 25 | 45.01 ± 1.30 | 27.53 ± 0.04 | 42.64 ± 22.47 |
| 1N PMF | 50 | 43.87 ± 0.76 | 31.70 ± 2.69 | 24.66 ± 1.62** |
| 1N PMF | 100 | 45.54 ± 9.96 | 33.39 ± 13.21 | 21.08 ± 3.25** |
| 6N PMF | 5 | 67.11 ± 1.53* | 19.17 ± 1.66 | 13.83 ± 0.13* |
| 6N PMF | 10 | 25.44 ± 4.07 | 33.98 ± 0.50 | 40.60 ± 4.57* |
| 6N PMF | 25 | 58.10 ± 0.93 | 22.63 ± 0.42 | 19.28 ± 1.35* |
| 6N PMF | 50 | 63.53 ± 0.23* | 22.78 ± 0.49 | 13.70 ± 0.71* |
| 6N PMF | 100 | 59.12 ± 1.12* | 31.87 ± 1.44 | 9.02 ± 0.32* |
| OPE | 5 | 62.36 ± 0.66* | 34.45 ± 3.54 | 3.20 ± 4.19 |
| OPE | 10 | 63.81 ± 0.52** | 32.97 ± 4.99 | 3.24 ± 4.46 |
| OPE | 25 | 75.25 ± 1.20** | 24.70 ± 1.13 | 0.05 ± 0.07 |
| OPE | 50 | 70.38 ± 1.29** | 29.40 ± 1.11 | 0.23 ± 0.18 |
| OPE | 100 | 65.44 ± 1.78* | 34.36 ± 2.07 | 0.26 ± 0.22 |

*P < 0.05,
**P < 0.01,
***P < 0.001

TABLE 7
AGS Cell Cycle Distributions

| | Concen- | | | |
|---|---|---|---|---|
| | tration | Cell Cycle Distribution | | |
| | (μM) | G0/G1 | S | G2/M |
| Control | — | 55.65 ± 1.61 | 32.94 ± 2.43 | 11.41 ± 1.28 |
| Nobiletin | 5 | 58.67 ± 0.61 | 27.84 ± 1.94 | 13.50 ± 1.34 |
| Nobiletin | 10 | 59.74 ± 0.36 | 26.70 ± 0.87 | 13.57 ± 1.23 |
| Nobiletin | 25 | 59.56 ± 1.98 | 26.95 ± 2.33 | 13.50 ± 0.35 |
| Nobiletin | 50 | 56.45 ± 0.03 | 28.31 ± 0.36 | 15.25 ± .33 |
| Nobiletin | 100 | 46.13 ± 4.96 | 28.33 ± 3.86 | 15.54 ± 1.10 |
| 1N PMF | 5 | 59.22 ± 0.08 | 47.95 ± 1.05 | 12.84 ± 1.14 |
| 1N PMF | 10 | 58.20 ± 0.66 | 28.70 ± 0.11 | 13.10 ± 0.55 |
| 1N PMF | 25 | 53.97 ± 3.19 | 18.07 ± 0.93 | 27.97 ± 2.26* |
| 1N PMF | 50 | 55.06 ± 5.29 | 9.84 ± 13.91 | 35.01 ± 8.48 |
| 1N PMF | 100 | 47.79 ± 5.98 | 51.72 ± 6.68 | 0.50 ± 0.71* |
| 6N PMF | 5 | 53.72 ± 4.45 | 2.96 ± 4.18 | 43.33 ± 0.28*** |
| 6N PMF | 10 | 44.46 ± 0.97 | 3.55 ± 5.02 | 52.00 ± 4.05*** |
| 6N PMF | 25 | 45.10 ± 0.24 | 33.86 ± 1.82 | 21.05 ± 1.58* |
| 6N PMF | 50 | 54.66 ± 1.21 | 23.46 ± 4.56 | 21.89 ± 3.36 |
| 6N PMF | 100 | 58.10 ± 1.21 | 17.23 ± 1.09 | 24.68 ± 0.12** |
| OPE | 5 | 59.24 ± 0.42 | 28.49 ± 2.69 | 12.27 ± 2.28 |
| OPE | 10 | 60.37 ± 1.26 | 27.06 ± 1.82 | 12.58 ± 0.56 |
| OPE | 25 | 66.00 ± 0.08** | 21.67 ± 0.11 | 12.34 ± 0.04 |
| OPE | 50 | 59.73 ± 1.03 | 22.65 ± 4.12 | 17.62 ± 3.08 |
| OPE | 100 | 51.07 ± 2.67 | 47.34 ± 3.51* | 0.59 ± 0.83 |

*P < 0.05,
**P < 0.01,
***P < 0.001

Anti-Inflammatory Activity.

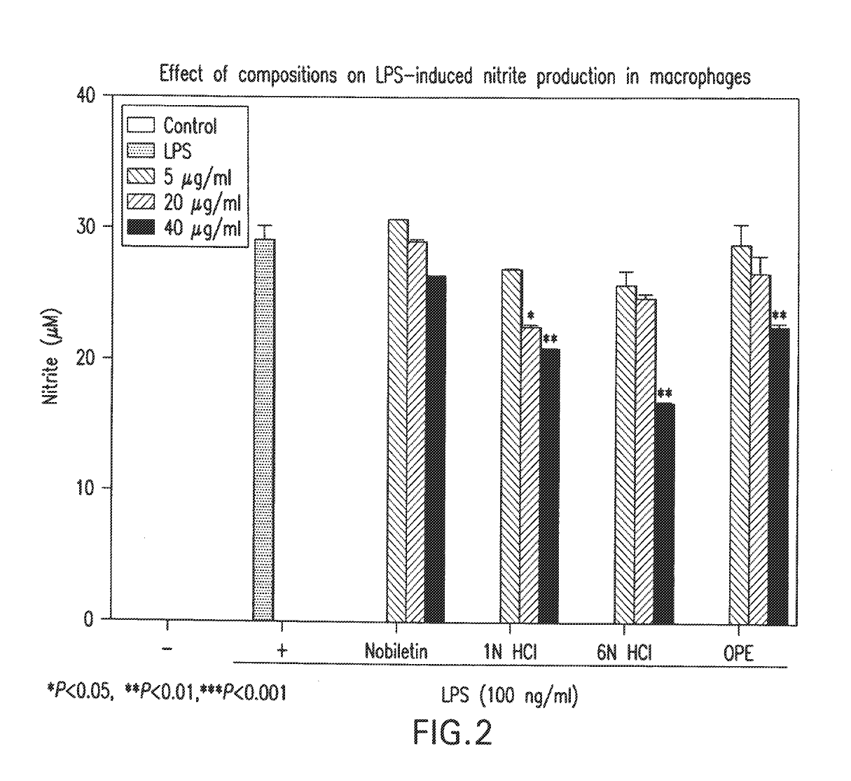

Anti-inflammatory activities of the hydrolyzed PMF-enriched compositions, nobiletin and 70% PMF OPE were studied using the protocol for the nitrite assay described in Section 8.4. Both the 1 N PMF and 6 N PMF hydrolyzed PMF-enriched compositions showed greater anti-inflammatory activity than either nobiletin or 70% PMF OPE, as seen in FIG. 2. These results demonstrate that hydroxylated PMF-enriched compositions are more potent anti-inflammatory compositions that nobiletin alone or OPE having a 70% predominantly non-hydroxylated PMF fraction.

### 8.6. Example 6

This examples compares anti-inflammatory and anti-cancer activities of individual hydroxylated PMFs against each other and against individual non-hydroxylated PMFs. Eleven compounds were used, as shown in Table 8 below, which are referred to throughout this example by the one letter designation indicated in the table.

TABLE 8

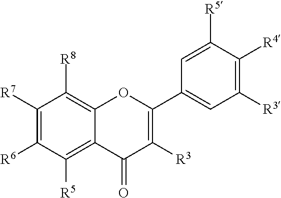

| | PMF | R³ | R⁵ | R⁶ | R⁷ | R⁸ | R³′ | R⁴′ | R⁵′ |
|---|---|---|---|---|---|---|---|---|---|
| A | nobiletin | H | OMe | OMe | OMe | OMe | OMe | OMe | H |
| B | 3-hydroxy-5,6,7,8,3′,4- | OH | OMe | OMe | OMe | OMe | OMe | OMe | H |
| | hexamethoxyflavone | | | | | | | | |
| C | 5-hydroxy-3,6,7,8,3′,4′- | OMe | OH | OMe | OMe | OMe | OMe | OMe | H |
| | hexamethoxyflavone | | | | | | | | |
| D | 3,5,6,7,8,3,4- | OMe | OMe | OMe | OMe | OMe | OMe | OMe | H |
| | heptamethoxyflavone | | | | | | | | |
| E | 5-hydroxy-6,7,8,3′,4′,5′- | H | OH | OMe | OMe | OMe | OMe | OMe | OMe |
| | hexamethoxyflavone | | | | | | | | |
| F | 5,6,7,8,3′,4′,5′- | H | OMe | OMe | OMe | OMe | OMe | OMe | OMe |
| | heptamethoxyflavone | | | | | | | | |
| G | 4′-hydroxy-5,6,7,8,3′- | H | OMe | OMe | OMe | OMe | OMe | OH | H |
| | pentamethoxyflavone | | | | | | | | |
| H | 3′,4′-dihydroxy-5,6,7,8- | H | OMe | OMe | OMe | OMe | OH | OH | H |
| | tetramethoxyflavone | | | | | | | | |
| I | 3′-hydroxy-5,6,7,8,4′- | H | OMe | OMe | OMe | OMe | OH | OMe | H |
| | pentamethoxyflavone | | | | | | | | |
| J | 5-hydroxy-6,7,8,3′,4′- | H | OH | OMe | OMe | OMe | OMe | OMe | H |
| | pentamethoxyflavone | | | | | | | | |
| K | 5,3′-dihydroxy-6,7,8,4′- | H | OH | OMe | OMe | OMe | OH | OMe | H |
| | tetramethoxyflavone | | | | | | | | |

Cancer Cell Antiproliferative and Apoptosis-Inducing Activities.

The PMF compounds were assayed in vitro for cell growth inhibition and for their ability to induce apoptosis in various human cancer cells, including HL-60, AGS, COLO 205, and HT-29 cells, using methods described in Section 8.4 above. Antiproliferative (IC₅₀) and apoptosis-inducing activity (AC₅₀) of PMFs in the HL-60, AGS, COLO 205, and HT-29 cells are reported in Table 9, where *P<0.05, **P<0.01 and ***P<0.001 indicate statistically significant differences from the control group.

TABLE 9
Antiproliferative (IC₅₀) and apoptosis-inducing (AC₅₀) activities of PMFs

| | HL-60 | | AGS | | COLO205 | | HT-29 | |
|---|---|---|---|---|---|---|---|---|
| | IC₅₀ | AC₅₀ | IC₅₀ | AC₅₀ | IC₅₀ | AC₅₀ | IC₅₀ | AC₅₀ |
| Cmpd | μM | μM | μM | μM | μM | μM | μM | μM |
| A | >100 | >100 | >100 | >100 | 38.02 ± 1.40*** | >100 | >100 | >100 |
| B | >100 | 56.51 ± 2.39*** | 72.31 ± 7.91* | >100 | 23.28 ± 1.56*** | >100 | 63.63 ± 0.49*** | >100 |
| C | 8.45 ± 1.6*** | 63.65 ± 0.61*** | 17.01 ± 0.21*** | 18.86 ± 0.41*** | 67.02 ± 1.40*** | >100 | 77.38 ± 1.82** | >100 |
| D | 54.39 ± 0.9*** | >100 | >100 | >100 | 62.5 ± 1.81*** | >100 | >100 | >100 |
| E | >100 | >100 | >100 | >100 | >100 | >100 | >100 | >100 |
| F | 47.03 ± 4.00*** | >100 | >100 | >100 | 92.59 ± 14.96 | >100 | 96.36 ± 8.66 | >100 |
| G | 47.41 ± 3.64*** | 87.10 ± 7.83 | 70.48 ± 9.97* | >100 | 83.71 ± 4.96* | >100 | 45.82 ± 5.06*** | >100 |
| H | 71.18 ± 1.93** | >100 | 69.33 ± 4.23** | >100 | 90.14 ± 11.88 | >100 | 74.72 ± 0.20*** | >100 |
| I | 52.72 ± 0.22*** | 94.62 ± 1.50 | 65.75 ± 0.41*** | >100 | 75.96 ± 1.88*** | >100 | >100 | >100 |
| J | >100 | >100 | >100 | >100 | >100 | >100 | >100 | >100 |
| K | 10.02 ± 1.33*** | >100 | 9.61 ± 0.22 | 69.95 ± 1.40*** | 11.15 ± 3.63*** | >100 | 40.02 ± 6.16*** | >100 |

Overall, these results demonstrate that PMFs vary in their abilities in inhibiting proliferation and inducing apoptosis. In many instances individual hydroxylated PMFs showed greater effectiveness in their activity to inhibit proliferation and/or induce apoptosis than nonhydroxylated PMFs. For instance, these data indicate that compound C is an effective anti-proliferative agent in HL-60 and AGS cells and has potent apoptotic-inducing activity in AGS carcinoma cells. Compound K is effective as an anti-proliferative agent in HL-60, AGS, COLO 205, and HT-29 cells with IC₅₀ values of 10.02, 9.61, 11.15, and 40.02 μM, respectively. Compound G is effective as an anti-proliferative agent in HT-29 cells with IC₅₀ of 45.82 μM.

Cell Cycle Distribution.

Compounds A-K were evaluated in terms of their effects in arresting cell cycle progression by observing cell cycle phase distribution in cells treated with the compounds. Cells were treated with 5, 10, 25, 50, and 100 μM of one of compound A-K or DMSO (control) for 24 h and subjected to flow cytometric analysis after staining their DNA, as described above. Results of the effects of PMFs on cell cycle distribution in human leukemia HL-60 cells are shown in Table 10.

TABLE 10
HL-60 Cell Cycle Distributions

| | Concen- | | | |
|---|---|---|---|---|
| | tration | Cell Cycle Distribution | | |
| | (μM) | G0/G1 | S | G2/M |
| Control | — | 36.69 ± 0.78 | 51.44 ± 0.07 | 11.88 ± 0.64 |
| A | 5 | 41.42 ± 0.14* | 50.73 ± 0.42 | 8.15 ± 0.49 |
| A | 10 | 50.36 ± 1.06** | 46.88 ± 0.28 | 2.75 ± 0.57 |
| A | 25 | 66.18 ± 0.71*** | 31.06 ± 0.99 | 2.77 ± 0.85 |
| A | 50 | 64.40 ± 0.49*** | 27.93 ± 0.35 | 7.67 ± 0.57 |
| A | 100 | 62.05 ± 0.14** | 30.60 ± 0.35 | 7.35 ± 0.14 |
| B | 5 | 42.63 ± 0.85* | 47.38 ± 0.42 | 9.99 ± 1.70 |
| B | 10 | 40.76 ± 0.28* | 59.01 ± 0.78* | 0.23 ± 0.42 |
| B | 25 | 47.74 ± 0.71** | 52.26 ± 0.64 | 0.00 ± 0.64 |
| B | 50 | 41.48 ± 0.85* | 58.52 ± 0.42* | 0.00 ± 0.57 |
| B | 100 | 49.63 ± 0.28** | 43.86 ± 0.21 | 0.51 ± 0.64 |
| C | 5 | 70.99 ± 0.49*** | 28.07 ± 0.99 | 0.95 ± 0.35 |
| C | 10 | 78.47 ± 0.64*** | 1.64 ± 0.42 | 19.89 ± 1.06** |
| C | 25 | 40.12 ± 0.64 | 39.51 ± 0.57 | 20.37 ± 0.57** |
| C | 50 | 51.42 ± 1.13** | 36.94 ± 0.85 | 11.64 ± 1.06 |
| C | 100 | 50.23 ± 1.91* | 31.03 ± 1.06 | 18.74 ± 1.56* |
| D | 5 | 50.66 ± 1.20** | 44.46 ± 0.49 | 4.88 ± 0.49 |
| D | 10 | 52.79 ± 0.99** | 42.12 ± 0.49 | 5.09 ± 0.35 |
| D | 25 | 57.63 ± 0.14** | 42.28 ± 0.57 | 0.09 ± 0.71 |
| D | 50 | 80.79 ± 0.71*** | 19.00 ± 0.78 | 0.21 ± 0.42 |
| D | 100 | 84.06 ± 0.57*** | 15.47 ± 1.06 | 0.47 ± 0.21 |
| E | 5 | 40.16 ± 1.10 | 53.83 ± 0.54 | 6.01 ± 1.64 |
| E | 10 | 40.43 ± 2.14 | 54.01 ± 0.98 | 5.57 ± 1.17 |
| E | 25 | 41.35 ± 2.50 | 52.38 ± 1.46 | 6.27 ± 1.05 |
| E | 50 | 37.64 ± 1.09 | 54.64 ± 0.49 | 7.73 ± 0.62 |
| E | 100 | 42.95 ± 0.11 | 49.79 ± 0.95 | 7.27 ± 0.84 |
| F | 5 | 42.11 ± 0.37 | 47.34 ± 1.53 | 10.56 ± 1.16 |
| F | 10 | 48.35 ± 1.83 | 41.07 ± 0.37 | 10.59 ± 1.46 |
| F | 25 | 57.82 ± 1.51* | 34.90 ± 0.03 | 7.28 ± 1.54 |
| F | 50 | 60.39 ± 0.23** | 31.18 ± 0.30 | 8.44 ± 0.54 |
| F | 100 | 63.66 ± 2.33* | 31.43 ± 1.87 | 4.92 ± 0.46 |
| G | 5 | 41.79 ± 0.80 | 53.41 ± 5.93 | 4.80 ± 6.73 |
| G | 10 | 43.26 ± 2.02 | 44.15 ± 2.12 | 12.61 ± 0.11** |
| G | 25 | 55.71 ± 0.15** | 35.90 ± 0.01 | 8.39 ± 0.16 |
| G | 50 | 52.35 ± 1.09 | 35.48 ± 4.91 | 12.18 ± 3.83 |
| G | 100 | 65.44 ± 5.42* | 23.35 ± 13.67 | 11.21 ± 8.27 |
| H | 5 | 49.78 ± 1.80 | 45.85 ± 6.80 | 4.37 ± 5.00 |
| H | 10 | 49.29 ± 1.53 | 47.95 ± 5.43 | 2.76 ± 3.90 |
| H | 25 | 52.82 ± 0.15* | 41.68 ± 0.93 | 5.49 ± 1.11 |
| H | 50 | 62.04 ± 1.19** | 27.27 ± 0.31 | 10.70 ± 0.89 |
| H | 100 | 61.15 ± 7.03 | 12.91 ± 15.34 | 25.94 ± 8.32 |
| I | 5 | 44.51 ± 2.37 | 46.27 ± 2.21 | 9.23 ± 0.16 |
| I | 10 | 43.22 ± 1.85 | 44.86 ± 2.49 | 11.92 ± 0.64 |
| I | 25 | 50.55 ± 0.97 | 38.71 ± 1.38 | 10.75 ± 0.41 |
| I | 50 | 50.23 ± 1.94 | 43.41 ± 3.62 | 6.36 ± 1.69 |
| I | 100 | 65.54 ± 4.07* | 21.31 ± 15.55 | 13.24 ± 11.61 |
| J | 5 | 53.10 ± 0.46 | 38.96 ± 0.51 | 7.94 ± 0.06 |
| J | 10 | 51.74 ± 1.57 | 39.20 ± 2.10 | 9.06 ± 0.54 |
| J | 25 | 51.30 ± 1.68 | 40.50 ± 2.87 | 8.20 ± 1.19 |
| J | 50 | 49.22 ± 0.13 | 42.86 ± 0.48 | 7.93 ± 0.36 |
| J | 100 | 51.29 ± 0.74 | 41.27 ± 0.09 | 7.45 ± 0.83 |
| K | 5 | 0.56 ± 0.76 | 60.54 ± 4.90 | 35.91 ± 0.11*** |
| K | 10 | 0.38 ± 0.06 | 45.22 ± 4.83 | 54.41 ± 4.77*** |
| K | 25 | 0.68 ± 0.42 | 50.23 ± 3.08 | 49.12 ± 2.64*** |
| K | 50 | 2.63 ± 2.50 | 52.33 ± 2.51 | 45.05 ± 0.01*** |
| K | 100 | 3.18 ± 2.62 | 66.21 ± 1.72* | 30.61 ± 4.64** |

*P < 0.05,
**P < 0.01,
***P < 0.001

Table 10 shows that non-hydroxylated compounds A, D, F, and hydroxylated compounds C and H in concentrations 25-100 μM caused a significant G1 arrest at the expense of S and G2/M phase cell population following 24 h treatment in HL-60 cells. In the case of 25 μM compound C this decrease in G1 arrest was accompanied by an increase in G2/M phase cell population at 24 h of treatment.

Similar cell cycle phase distribution studies were performed in AGS, COLO205, and HT-29 cells in response to individual PMFs, with results reported in Tables 11, 12 and 13, respectively.

TABLE 11
Human Gastric Carcinoma AGS Cell Cycle Distributions

| | Concen- | | | |
|---|---|---|---|---|
| | tration | Cell Cycle Distribution | | |
| | (μM) | G0/G1 | S | G2/M |
| Control | — | 46.1 ± 0.71 | 50.7 ± 0.64 | 4.6 ± 0.49 |
| A | 5 | 44.7 ± 0.71 | 54.4 ± 0.64* | 0.4 ± 0.57 |
| A | 10 | 47.1 ± 0.28 | 49.1 ± 0.64 | 5.0 ± 0.64 |
| A | 25 | 52.9 ± 1.06* | 43.7 ± 0.42 | 2.8 ± 0.35 |
| A | 50 | 53.8 ± 0.64** | 41.3 ± 0.92 | 6.6 ± 0.69 |
| A | 100 | 66.9 ± 0.14*** | 31.0 ± 0.64 | 3.2 ± 0.57 |
| B | 5 | 47.1 ± 1.27 | 45.8 ± 1.20 | 5.6 ± 0.28 |
| B | 10 | 46.7 ± 0.92 | 44.1 ± 0.57 | 10.4 ± 0.07** |
| B | 25 | 39.8 ± 0.78 | 54.2 ± 0.28* | 6.5 ± 0.07 |
| B | 50 | 43.1 ± 1.56 | 51.2 ± 1.06 | 7.4 ± 0.42 |
| B | 100 | 46.8 ± 0.28 | 44.5 ± 0.14 | 9.3 ± 0.42** |
| C | 5 | 45.5 ± 1.06 | 45.2 ± 0.35 | 9.9 ± 0.07** |
| C | 10 | 29.6 ± 1.77 | 26.1 ± 0.85 | 22.0 ± 0.78** |
| C | 25 | 20.6 ± 0.42 | 31.3 ± 0.49 | 47.3 ± 1.13*** |
| C | 50 | 18.2 ± 0.42 | 50.1 ± 1.20 | 32.7 ± 0.57*** |
| C | 100 | 17.0 ± 0.07 | 52.0 ± 0.85 | 32.4 ± 0.78*** |
| D | 5 | 37.6 ± 0.92 | 62.4 ± 0.85** | 0.4 ± 0.57 |
| D | 10 | 44.2 ± 0.14 | 55.2 ± 0.42* | 1.3 ± 0.42 |
| D | 25 | 46.6 ± 0.35 | 48.8 ± 0.42 | 4.7 ± 0.14 |
| D | 50 | 48.0 ± 0.14 | 45.7 ± 0.35 | 6.9 ± 0.42* |
| D | 100 | 63.2 ± 0.78** | 33.0 ± 0.49 | 4.0 ± 0.14 |
| Control | — | 58.22 ± 1.18 | 25.45 ± 2.50 | 16.34 ± .32 |
| E | 5 | 55.70 ± 0.14 | 28.42 ± 0.75 | 15.88 ± 0.89 |
| E | 10 | 56.96 ± 3.14 | 27.05 ± 2.60 | 16.00 ± 0.55 |
| E | 25 | 56.32 ± 2.62 | 29.25 ± 0.18 | 14.40 ± 2.38 |
| E | 50 | 54.53 ± 0.30 | 29.79 ± 1.10 | 15.69 ± 1.41 |
| E | 100 | 55.68 ± 0.46 | 30.82 ± 0.29 | 13.51 ± 0.17 |
| F | 5 | 58.22 ± 1.10 | 27.02 ± 0.17 | 14.76 ± 0.94 |
| F | 10 | 62.41 ± 6.13 | 23.04 ± 5.55 | 14.56 ± 0.58 |
| F | 25 | 62.24 ± 0.71 | 22.57 ± 2.21 | 15.34 ± 1.30 |
| F | 50 | 65.87 ± 0.93* | 21.08 ± 1.90 | 13.05 ± 0.98 |
| F | 100 | 64.22 ± 5.56 | 21.86 ± 4.50 | 13.92 ± 1.06 |
| G | 5 | 55.39 ± 0.47 | 27.86 ± 1.32 | 16.76 ± 0.86 |
| G | 10 | 60.42 ± 1.28 | 22.79 ± 0.47 | 16.80 ± 1.75 |
| G | 25 | 68.29 ± 0.95** | 17.33 ± 0.80 | 14.39 ± 0.16 |
| G | 50 | 62.27 ± 0.06 | 14.78 ± 1.83 | 22.98 ± 1.90* |
| G | 100 | 50.27 ± 0.52 | 22.88 ± 2.38 | 26.85 ± 1.85* |
| H | 5 | 62.78 ± 2.93 | 20.66 ± 1.00 | 16.57 ± 1.93 |
| H | 10 | 61.72 ± 2.76 | 20.89 ± 0.00 | 17.39 ± 2.76 |
| H | 25 | 63.91 ± 0.92 | 19.28 ± 0.12 | 16.82 ± 1.04 |
| H | 50 | 63.96 ± 1.65 | 19.31 ± 0.51 | 16.74 ± 2.15 |
| H | 100 | 68.04 ± 1.34* | 9.27 ± 1.46 | 22.69 ± 0.13 |
| I | 5 | 57.25 ± 0.28 | 26.74 ± 0.49* | 16.01 ± 0.21 |
| I | 10 | 58.27 ± 0.96 | 23.79 ± 2.03 | 17.94 ± 1.07 |
| I | 25 | 49.07 ± 1.22 | 34.79 ± 1.90* | 16.15 ± 0.69 |
| I | 50 | 62.72 ± 1.27 | 36.70 ± 2.00* | 0.59 ± 0.74 |
| I | 100 | 69.35 ± 0.70** | 18.01 ± 0.14 | 12.65 ± 0.84 |
| J | 5 | 58.65 ± 1.77 | 27.84 ± 0.56 | 13.51 ± 1.20 |
| J | 10 | 57.07 ± 1.02 | 29.09 ± 1.54 | 13.84 ± 0.52 |
| J | 25 | 58.84 ± 4.84 | 27.17 ± 0.64 | 14.00 ± 4.20 |
| J | 50 | 52.82 ± 3.22 | 31.45 ± 3.31 | 15.74 ± 0.11 |
| J | 100 | 58.11 ± 1.11 | 29.24 ± 1.28 | 12.71 ± 0.25 |
| K | 5 | 51.75 ± 0.73 | 34.61 ± 0.91* | 13.65 ± 0.18 |
| K | 10 | 22.20 ± 1.56 | 26.69 ± 0.91 | 48.12 ± 1.35** |
| K | 25 | 42.63 ± 0.05 | 22.81 ± 0.51 | 34.57 ± 0.62** |
| K | 50 | 43.90 ± 2.37 | 20.44 ± 0.08 | 35.67 ± 2.29** |
| K | 100 | 50.77 ± 1.01 | 18.04 ± 1.45 | 31.20 ± 0.43** |

*P < 0.05,
**P < 0.01,
***P < 0.001

TABLE 12
Human Colon Carcinoma COLO205 Cell Cycle Distributions

| | Concen- | | | |
|---|---|---|---|---|
| | tration | Cell Cycle Distribution | | |
| | (μM) | G0/G1 | S | G2/M |
| Control | — | 61.93 ± 0.74 | 31.30 ± 1.77 | 6.78 ± 1.03 |
| A | 5 | 63.63 ± 0.53 | 34.80 ± 0.57 | 1.58 ± 0.04 |
| A | 10 | 67.25 ± 0.38* | 32.50 ± 0.13 | 0.25 ± 0.25 |
| A | 25 | 69.94 ± 1.02* | 27.42 ± 1.09 | 0.64 ± 0.07 |
| A | 50 | 82.40 ± 0.63** | 17.50 ± 0.45 | 0.29 ± 0.08 |
| A | 100 | 65.60 ± 0.65 | 28.21 ± 0.60 | 6.20 ± 1.25 |
| B | 5 | 63.59 ± 0.48 | 35.53 ± 0.46 | 0.89 ± 0.94 |
| B | 10 | 63.12 ± 0.20 | 31.25 ± 0.48 | 5.63 ± 0.28 |
| B | 25 | 64.96 ± 0.99 | 34.63 ± 0.68 | 0.41 ± 0.31 |
| B | 50 | 63.32 ± 0.52 | 36.39 ± 0.62 | 0.31 ± 0.11 |
| B | 100 | 78.51 ± 0.63** | 17.51 ± 1.58 | 3.99 ± 0.95 |
| C | 5 | 63.55 ± 0.99 | 30.29 ± 0.61 | 6.16 ± 0.37 |
| C | 10 | 75.57 ± 0.83** | 18.07 ± 0.67 | 6.37 ± 1.50 |
| C | 25 | 74.27 ± 0.83** | 25.30 ± 0.63 | 0.43 ± 0.20 |
| C | 50 | 58.30 ± 0.57 | 25.00 ± 1.16 | 16.70 ± 0.59** |
| C | 100 | 51.84 ± 0.31 | 29.13 ± 0.70 | 19.04 ± 0.39** |
| D | 5 | 60.81 ± 0.62 | 39.16 ± 0.67** | 0.04 ± 0.05 |
| D | 10 | 60.58 ± 0.57 | 39.02 ± 0.61** | 0.40 ± 0.04 |
| D | 25 | 61.91 ± 0.35 | 28.82 ± 0.23 | 9.28 ± 0.59 |
| D | 50 | 68.87 ± 1.53* | 25.03 ± 1.12 | 6.10 ± 2.64 |
| D | 100 | 75.17 ± 0.05** | 19.12 ± 0.69 | 5.72 ± 0.64 |
| E | 5 | 67.66 ± 0.19 | 23.60 ± 0.03 | 8.75 ± 0.17 |
| E | 10 | 67.48 ± 0.59 | 24.24 ± 0.11 | 8.29 ± 0.48 |
| E | 25 | 67.40 ± 0.49 | 25.02 ± 0.70 | 7.59 ± 1.18 |
| E | 50 | 69.49 ± 2.62 | 23.93 ± 1.80 | 6.59 ± 0.82 |
| E | 100 | 69.99 ± 0.89* | 24.50 ± 0.00 | 5.52 ± 0.90 |
| F | 5 | 68.30 ± 1.36 | 23.00 ± 0.80 | 8.71 ± 0.55* |
| F | 10 | 71.96 ± 0.24*** | 19.67 ± 0.67 | 8.38 ± 0.43* |
| F | 25 | 72.22 ± 0.54** | 17.98 ± 0.74 | 9.80 ± 0.20** |
| F | 50 | 76.39 ± 0.85** | 15.60 ± 1.35 | 8.02 ± 0.50 |
| F | 100 | 78.64 ± 0.34*** | 12.71 ± 0.52 | 8.65 ± 0.19** |
| G | 5 | 67.93 ± 0.74 | 27.36 ± 0.56 | 4.71 ± 0.18 |
| G | 10 | 72.87 ± 2.37 | 22.64 ± 2.28 | 4.51 ± 0.08 |
| G | 25 | 73.74 ± 0.51** | 19.80 ± 0.42 | 6.47 ± 0.93 |
| G | 50 | 73.90 ± 0.41** | 17.94 ± 0.17 | 8.17 ± 0.23** |
| G | 100 | 73.05 ± 0.30** | 19.35 ± 0.76 | 7.61 ± 0.46* |
| H | 5 | 67.26 ± 0.45 | 24.58 ± 0.15 | 8.17 ± 0.60 |
| H | 10 | 66.67 ± 0.16 | 27.16 ± 0.46 | 6.18 ± 0.63 |
| H | 25 | 66.99 ± 1.32 | 26.13 ± 1.11 | 6.89 ± 0.21 |
| H | 50 | 64.49 ± 1.00 | 26.57 ± 2.12 | 8.94 ± 1.12 |
| H | 100 | 66.50 ± 0.06 | 26.53 ± 0.08 | 6.98 ± 0.02 |
| I | 5 | 64.51 ± 1.26 | 29.03 ± 1.61 | 6.46 ± 0.35 |
| I | 10 | 64.14 ± 1.71 | 30.32 ± 1.30 | 5.54 ± 0.41 |
| I | 25 | 67.09 ± 0.57 | 27.48 ± 0.62 | 5.43 ± 1.17 |
| I | 50 | 68.03 ± 1.51 | 27.15 ± 0.04 | 4.83 ± 1.48 |
| I | 100 | 83.31 ± 1.06** | 16.68 ± 1.04 | 0.02 ± 0.02 |
| J | 5 | 56.75 ± 1.52 | 35.85 ± 1.22 | 7.41 ± 0.30 |
| J | 10 | 59.63 ± 0.88 | 31.99 ± 1.85 | 7.07 ± 0.86 |
| J | 25 | 63.97 ± 0.56 | 29.65 ± 1.61 | 6.38 ± 1.05 |
| J | 50 | 64.90 ± 1.44 | 28.69 ± 1.57 | 6.41 ± 0.13 |
| J | 100 | 62.39 ± 2.98 | 30.57 ± 2.84 | 7.05 ± 0.13 |
| K | 5 | 57.68 ± 0.42 | 34.52 ± 0.21 | 7.08 ± 0.20 |
| K | 10 | 65.81 ± 0.83 | 32.44 ± 2.28 | 1.75 ± 0.47 |
| K | 25 | 66.63 ± 1.51 | 20.06 ± 0.40 | 13.31 ± 1.91* |
| K | 50 | 42.30 ± 1.12 | 34.29 ± 0.29 | 23.42 ± 0.83** |
| K | 100 | 43.58 ± 2.29 | 33.57 ± 0.67 | 22.85 ± 2.96** |

*P < 0.05,
**P < 0.01,
***P < 0.001

TABLE 13
Human Colon Carcinoma HT-29 Cell Cycle Distributions

| | Concen- | | | |
|---|---|---|---|---|
| | tration | Cell Cycle Distribution | | |
| | (μM) | G0/G1 | S | G2/M |
| Control | — | 63.02 ± 0.17 | 27.76 ± 0.56 | 9.22 ± 0.74 |
| A | 5 | 63.67 ± 0.14 | 28.12 ± 0.52 | 8.22 ± 0.37 |
| A | 10 | 66.57 ± 0.66 | 26.98 ± 0.94 | 7.45 ± 0.28 |
| A | 25 | 69.67 ± 1.15* | 22.76 ± 1.15 | 7.58 ± 0.00 |
| A | 50 | 83.26 ± 0.46*** | 11.79 ± 1.13 | 4.97 ± 0.67 |
| A | 100 | 89.49 ± 0.57*** | 4.92 ± 0.00 | 5.60 ± 0.57 |
| B | 5 | 63.71 ± 1.80 | 29.8 ± 1.42 | 6.50 ± 0.37 |
| B | 10 | 69.27 ± 3.06 | 25.14 ± 2.21 | 5.60 ± 0.85 |
| B | 25 | 70.85 ± 1.84* | 24.8 ± 1.23 | 4.37 ± 0.62 |
| B | 50 | 70.11 ± 1.44* | 19.18 ± 0.96 | 10.73 ± 0.47* |
| B | 100 | 66.75 ± 1.06 | 24.45 ± 0.80 | 8.82 ± 0.26 |
| C | 5 | 65.24 ± 0.32 | 27.15 ± 0.21 | 7.62 ± 0.11 |
| C | 10 | 69.14 ± 0.14* | 24.28 ± 0.32 | 6.58 ± 0.18 |
| C | 25 | 80.09 ± 0.19*** | 15.54 ± 0.19 | 4.39 ± 0.37 |
| C | 50 | 69.85 ± 0.66* | 24.25 ± 1.15 | 5.91 ± 0.49 |
| C | 100 | 77.48 ± 0.74** | 18.59 ± 0.58 | 3.94 ± 1.32 |
| D | 5 | 67.79 ± 0.17** | 24.66 ± 0.06 | 7.56 ± 0.23 |
| D | 10 | 57.61 ± 0.69 | 30.98 ± 1.18 | 10.41 ± 0.45 |
| D | 25 | 76.11 ± 1.56** | 14.89 ± 1.21 | 9.04 ± 2.77 |
| D | 50 | 77.31 ± 2.82* | 20.49 ± 2.09 | 12.21 ± 0.74* |
| D | 100 | 80.66 ± 0.01*** | 15.57 ± 0.43 | 3.79 ± 0.43 |
| E | 5 | 57.96 ± 0.21 | 31.85 ± 0.14 | 10.19 ± 0.07 |
| E | 10 | 57.86 ± 0.31 | 32.14 ± 0.78 | 10.01 ± 0.46 |
| E | 25 | 59.20 ± 0.06 | 31.18 ± 0.74 | 9.63 ± 0.69 |
| E | 50 | 58.95 ± 0.95 | 31.79 ± 1.12 | 9.26 ± 0.16 |
| E | 100 | 61.79 ± 1.47 | 29.42 ± 0.24 | 8.79 ± 1.23 |
| F | 5 | 70.24 ± 0.52* | 22.66 ± 0.35 | 7.11 ± 0.17 |
| F | 10 | 76.34 ± 0.35** | 16.61 ± 0.06 | 7.06 ± 0.42 |
| F | 25 | 82.19 ± 0.16** | 10.98 ± 0.45 | 6.83 ± 0.61 |
| F | 50 | 88.64 ± 0.27*** | 5.11 ± 0.31 | 6.25 ± 0.58 |
| F | 100 | 89.85 ± 0.13*** | 4.10 ± 0.06 | 6.07 ± 0.06 |
| G | 5 | 56.92 ± 1.98 | 30.66 ± 2.43 | 12.42 ± 0.44* |
| G | 10 | 58.01 ± 1.22 | 32.03 ± 0.88 | 9.97 ± 2.10 |
| G | 25 | 74.38 ± 1.68* | 19.55 ± 1.59 | 6.18 ± 0.06 |
| G | 50 | 84.63 ± 1.16** | 7.39 ± 1.16 | 7.99 ± 0.01 |
| G | 100 | 75.28 ± 1.61* | 10.11 ± 1.82 | 14.63 ± 0.21** |
| H | 5 | 63.21 ± 1.82 | 29.26 ± 2.63 | 7.53 ± 0.81 |
| H | 10 | 64.87 ± 0.74 | 26.95 ± 0.64 | 8.19 ± 0.09 |
| H | 25 | 63.87 ± 0.92 | 27.68 ± 1.37 | 8.45 ± 0.46 |
| H | 50 | 63.10 ± 0.16 | 28.98 ± 0.69 | 7.94 ± 0.84 |
| H | 100 | 61.66 ± 1.44 | 33.37 ± 0.11* | 4.98 ± 1.55 |
| I | 5 | 60.78 ± 1.52 | 31.28 ± 0.81 | 7.95 ± 0.71 |
| I | 10 | 61.09 ± 1.06 | 31.02 ± 1.30 | 7.89 ± 0.23 |
| I | 25 | 60.90 ± 0.87 | 31.63 ± 0.23 | 7.48 ± 0.64 |
| I | 50 | 66.36 ± 0.88* | 23.81 ± 0.28 | 9.83 ± 0.61 |
| I | 100 | 74.18 ± 2.33* | 25.45 ± 2.72 | 0.37 ± 0.40 |
| J | 5 | 64.81 ± 0.41 | 25.68 ± 0.46 | 9.52 ± 0.05 |
| J | 10 | 62.87 ± 0.40 | 28.11 ± 0.57 | 9.02 ± 0.16 |
| J | 25 | 62.04 ± 1.24 | 29.51 ± 1.15 | 8.46 ± 0.09 |
| J | 50 | 60.39 ± 0.28 | 29.55 ± 0.52 | 9.93 ± 0.06 |
| J | 100 | 62.44 ± 1.00 | 29.67 ± 1.68 | 7.89 ± 0.69 |
| K | 5 | 60.57 ± 0.04 | 29.71 ± 0.94 | 9.87 ± 0.78 |
| K | 10 | 57.94 ± 0.50 | 30.55 ± 0.77 | 11.52 ± 0.27 |
| K | 25 | 50.58 ± 1.35 | 36.94 ± 1.45 | 12.49 ± 0.09 |
| K | 50 | 64.13 ± 0.83 | 22.73 ± 0.37 | 13.14 ± 0.47* |
| K | 100 | 47.16 ± 2.59 | 16.72 ± 1.73 | 36.12 ± 4.37** |

*P < 0.05,
**P < 0.01,
***P < 0.001

Compound C (5-100 μM) showed a decrease in G1 arrest accompanied by an increase in G2/M phase cell population at 24 h of treatment in AGS and COLO 205 cells. Compound A (50 μM) and compound I (100 μM) showed trends in G1 arrest at 24 h of treatment as observed in COLO 205 cells. As shown in Table 13, a 24 h exposure of HT-29 cells to compound A, C, D, F, G, and I (5-100 μM) resulted in significant accumulation of cells in G1 phase that was accompanied by a decrease in cells with G2/M phase. Treatment with compound K in the four types of human cells strongly potentiated the G2/M cell cycle arrest. Taken together, these data suggest considerable effect on the extent as well as nature of the cell cycle arrest in cancer cells when PMFs ring is modified by substitution at different position with different methoxyl and hydroxyl moieties. These data obtained support the conclusion that these PMFs impart anticancer activity to different extents in human cancer cells such as HL-60, AGS, COLO 205, and HT-29 cells.

Anti-Inflammatory Activity.

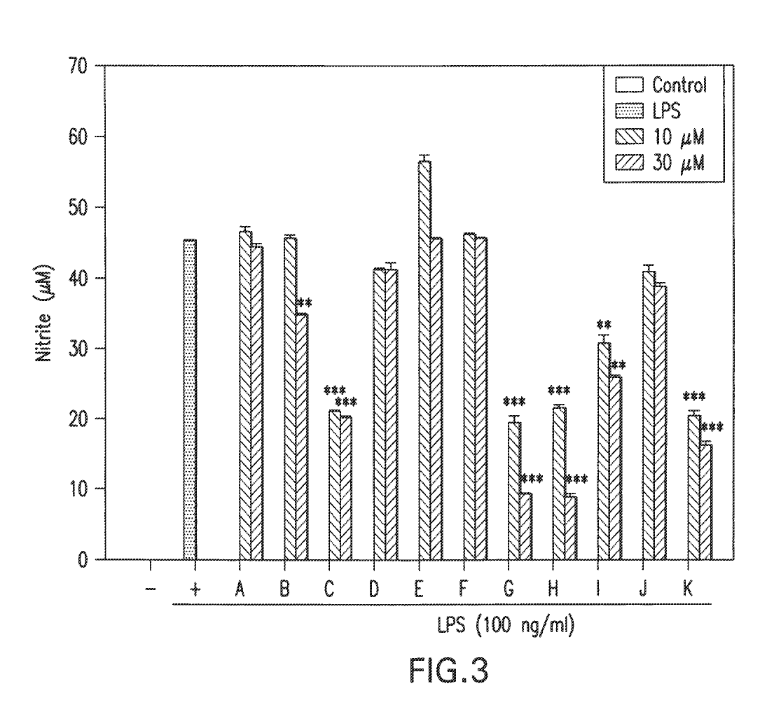

The anti-inflammatory effects of compounds A-K were tested by measuring nitrite production in in LPS-activated RAW 264.7 macrophages, as described in Section 8.4 above. Macrophages were treated with 100 ng/ml LPS alone or LPS and PMF (10 or 30 μM) for 24 h. At the end of incubation time, 100 μl of the culture medium was collected for the nitrite assay. Results are shown in FIG. 3.

The results indicate that when the PMFs are compared at a concentration of 30 μM, certain hydroxylated PMFs were the most effective at inhibiting LPS-induced nitrate production. The PMFs with inhibitory effects can be ranked according to their inhibitory potency as compound H>G>K>C>I>B, all hydroxylated PMFs. Among them, compound H and G are the most potent inhibitors for nitrite production in macrophages.

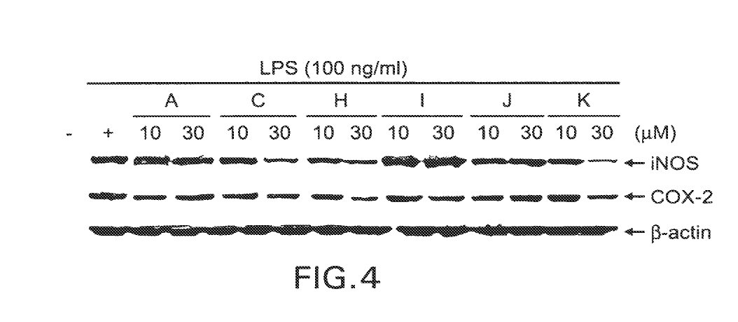

Western blot analysis of inhibition of LPS-induced iNOS and COX-2 protein expression by PMFs was analyzed, following procedures described above. Briefly, macrophage cells were treated with LPS (100 ng/ml) alone or with PMFs (10 or 30 μM) for 24 h. Equal amounts of total proteins (50 μg) were subjected to 10% SDS-PAGE, and expression of iNOS, COX-2 and β-actin protein was detected by Western blot using specific antibodies. As shown in FIG. 4, among the selected PMFs, compound K (30 μM) was the most potent inhibitor of iNOS expression in LPS-activated macrophages, while compound H (30 μM) was the most potent inhibitor of COX-2 expression.

Deletion and mutation analyses have demonstrated the transcription factor NFκB is involved in the activation of iNOS and COX-2 by LPS. To confirm whether selected PMFs inhibited NFκB binding activity in LPS-induced macrophages, assessment of luciferase expression in cells transiently transfected with NFκB-dependent luciferase reporter plasmid was assessed according to the procedures described above in Section 8.4.

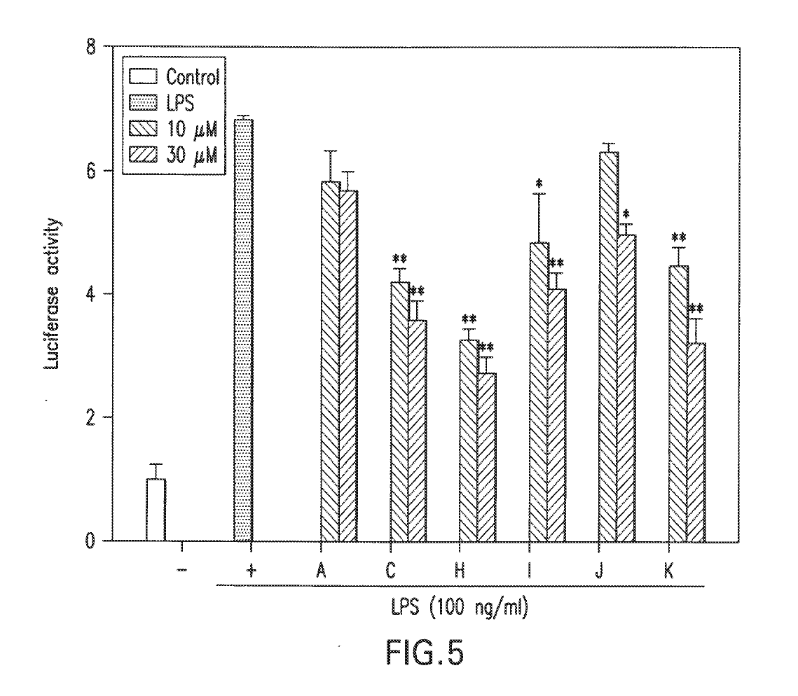

As shown in FIG. 5, compound H inhibited LPS-induced NFκB transcriptional activity in a concentration-dependent manner. At the concentration of 30 μM, compound H had the greatest inhibitory potency of PMFs tested, followed by compound K, compound C, compound I and J. These results indicate that compound H blocks LPS-induced NFκB activation by inhibiting IKK activity, which could perturb the degradation of IκBα and IκBβ. Inhibition of IκBα and IκBβ degradation is expected to lead to the inhibition of LPS-induced iNOS and COX-2 expression.

Taken together, these results demonstrate that hydroxylated PMFs have significant anti-inflammatory properties.

### 8.7. Example 7

This example demonstrates the anti-inflammatory activity of an exemplary hydroxylated PMF utilizing a COX-2 expression assay.

COX-2, an enzyme pivotal in the intracellular prostaglandin biosynthetic pathway, is rapidly upregulated during the course of inflammation, following cellular stresses, and in response to growth factors, tumor promoters, hormones, bacterial endotoxins and inflammatory cytokines. Assays that monitor COX-2 expression are art-recognized tools for the identification of inflammation modulators.

Bacterial lipopolysaccharide (LPS)-induced COX-2 expression was monitored in RAW264.7 macrophages, as previously described. Briefly, COX-2 expression was determined in untreated cells and cells treated with 100 ng/mL LPS either in the absence or the presence of 20 μM 5-hydroxy-3,6,7,8,3′,4′-hexamethoxyflavone. COX-2 mRNA levels were determined by real-time PCR where the values obtained were relative to expression of glyceraldehyde-3-phosphate dehydrogenase (G3PDH).

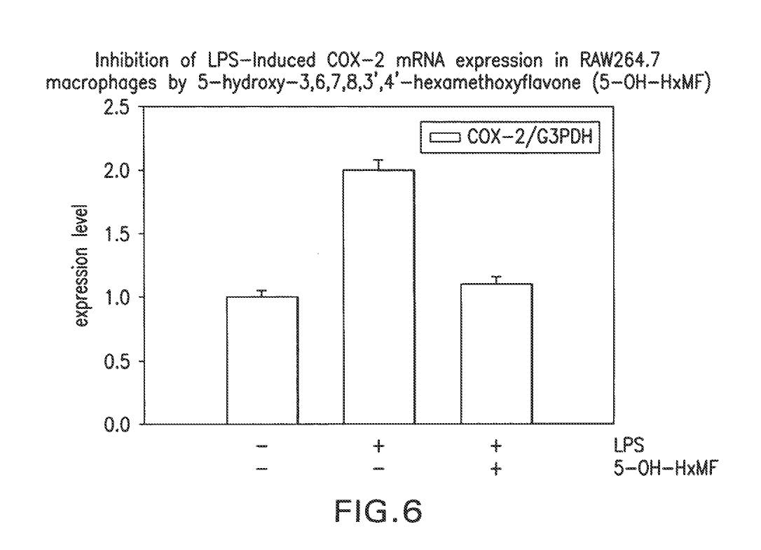

FIG. 6 provides experimental results demonstrating that 20 μM 5-hydroxy-3,6,7,8,3′,4′-hexamethoxyflavone inhibits LPS-induced COX-2 mRNA expression in macrophages. These data exemplify the anti-inflammatory activities of the compositions provided herein.

### 8.8. Example 8

This example demonstrates that PMFs induce Ca²⁺-mediated apoptosis in breast cancer cells. The compounds used, shown in Table 14 below, are referred to throughout this example by the numerical designation indicated in the table.

TABLE 14

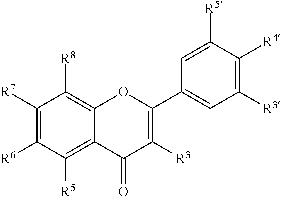

| | PMF | R³ | R⁵ | R⁶ | R⁷ | R⁸ | R³′ | R⁴′ | R⁵′ |
|---|---|---|---|---|---|---|---|---|---|
| 4 | 5-hydroxy- | OMe | OH | OMe | OMe | OMe | OMe | OMe | H |
| | 3,6,7,8,3′,4′- | | | | | | | | |
| | hexamethoxy- | | | | | | | | |
| | flavone | | | | | | | | |
| 5 | 3′-hydroxy- | H | OMe | OMe | OMe | H | OH | OMe | H |
| | 5,6,7,4′- | | | | | | | | |
| | tetramethoxy- | | | | | | | | |
| | flavone | | | | | | | | |
| 6 | 3,5,6,7,8,3′,4′- | OMe | OMe | OMe | OMe | OMe | OMe | OMe | H |
| | heptamethoxy- | | | | | | | | |
| | flavone | | | | | | | | |
| 7 | 5,6,7,3′,4′- | H | OMe | OMe | OMe | H | OMe | OMe | H |
| | pentamethoxy- | | | | | | | | |
| | flavone | | | | | | | | |

Sustained elevations in intracellular Ca²⁺ ([Ca²⁺]ᵢ) and a Ca²⁺-mediated, calpain/caspase-12-dependent signaling pathway have been shown to lead to apoptosis in breast cancer cells. See, e.g., Reed (2003) Cancer Cell 3:17-22; Sergeev (2004) Biochem. Biophys. Res. Commun. 321:462-467; Sergeev (2005) J. Steroid Biochem. Mol. Biol. 97:145-151; Mathiasen et al. (2002) J. Biol. Chem. 277:3078-30745.

### 8.8.1. Methods and Materials

Apoptosis Assay.

MCF-7 cells were maintained and assayed as described in Section 8.4 above. Apoptosis was evaluated by the plasma membrane and nuclear changes as previously described in Sergeev (2004) J. Steroid Biochem. Mol. Biol. 89-90:419-425. Briefly, An Annexin V assay (ALEXA FLUOR 488 Annexin V Assay Kit; Molecular Probes) was used for detection of the apoptotic plasma membrane (loss of membrane asymmetry due to phosphatidylserine translocation). Fluorescence (485 nm excitation, 530 nm emission) of the Annexin V-abeled cells grown in 96-well plates was measured in the FLx800 plate reader with KC software (BioTek) and expressed in fluorescence units per 1×10³ cells. Propidium iodide uptake was used to evaluate non-apoptotic and apoptotic cell death (the dye permeates plasma membrane and stain nucleic acids of both necrotic and the late apoptotic cells). Fluorescence (530 nm excitation, 620 nm emission) of the propidium iodide-labeled cells was also measured in the FLx800 reader. Additionally, HOECHST 33342 and ALEXA FLUOR 488 Annexin V were employed to visualize apoptotic nuclei (nuclear fragmentation) and the apoptotic plasma membrane, respectively. Fluorescence microscopy of HOECHST 33342- and Annexin V-labeled cells was performed as described below for the cellular Ca²⁺ and immunofluorescence imaging.

Intracellular Calcium Measurement.

Concentration of intracellular free Ca²⁺ ([Ca²⁺]ᵢ) was measured with Ca²⁺ indicators fura-2 and fluo-3, as described in detail previously, for example, in Sergeev (2004) J. Steroid Biochem. Mol. Biol. 89-90:419-425. For [Ca²⁺]ᵢ measurements with fluo-3, cells grown in the 96-well, black-wall plates were loaded with 2 μM of fluo-3/AM (Molecular Probes) in Dulbecco's PBS (D-PBS) supplemented with 0.1% DMSO for 40 min at 37° C. Fluorescence (485 nm excitation, 530 nm emission) was measured in the FLx800 plate reader, as described above. For [Ca²⁺]ᵢ measurements with fura-2, cells grown on coverslips were loaded with 1 μM of fura-2/AM (Molecular Probes) in D-PBS supplemented with 0.1% DMSO and 0.01% Pluronic F-127 for 40 min at 37° C. The dynamics of intracellular Ca²⁺ was assessed with cells in the microincubation chamber (37.0±0.2° C.) on a NIKON ECLIPSE TE-300 inverted microscope equipped for epifluorescence, ratiometric, digital imaging. The images were captured using SUPERFLUOR 40× 1.3 NA oil-immersion objective (Nikon) and COOLSNAPFX digital CCD camera (Photometrics), ratioed (340/380 nm excitation, 510 nm emission) on a pixel-by-pixel basis, and stored for analysis. Image analysis was performed using METAFLUOR 6.3 software (Molecular Devices/Universal Imaging).

To evaluate Ca²⁺ influx from the extracellular space, the Mn²⁺ entry rate, as a reporter of Ca²⁺ influx, was measured. The images were recorded at excitation of 360 nm (the fura-2 isosbestic point) and 2 mM of extracellular Mn²⁺. The rates of Ca²⁺ entry were estimated from the slope of the liner portion of curves of the fura-2 fluorescence quench by Mn²⁺. To evaluate Ca²⁺ release from the intracellular stores, cells grown on coverslips were placed in D-PBS, and the mobilizer of the endoplasmic reticulum Ca²⁺-stores, thapsigargin (1 μM), was added after recording the basal [Ca²⁺]ᵢ. The peak values of the [Ca²⁺]ᵢ increase, indicating the filling level of the Ca²⁺-stores, were measured with fura-2.

Calpain and Caspase-12 Activities Assays.

Calpain and caspase-12 activities were measured with the cell-permeable fluorogenic peptide substrates t-Boc-Leu-Met-CMAC (50 μM; CMAC, 7-amino-4-chloromethyl coumarin; Molecular Probes) and Ala-Thr-Ala-Asp-AFC (50 μM; AFC, 7-amino-4-trifluoromethyl coumarin; Caspase-12 Fluorometric Assay Kit, BioVision), respectively. See, e.g., Roser and Gores (2000) Meth. Mol. Biol. 144:2452-2466. Polyclonal antibodies directed against caspase-12 (BioVision) and monoclonal antibodies directed against the calpain small (cleaved) subunit (Chemicon) were used to evaluate activation of these proteases. For immunofluorescence labeling, the fixed and permeabilized cell preparations were pre-incubated with non-specific serum for 20 min, incubated for 1 h at 37° C. or overnight at 4° C. with the primary antibodies and 1 h at room temperature with secondary antibodies using ALEXA FLUOR-488 signal-amplification mouse antibodies (Molecular Probes) and FITC- or Texas Red-conjugated anti-rabbit and anti-mouse IgG (Molecular Probes). Fluorescence microscopy was carried out as described above for Ca²⁺ imaging. Image analysis and measurement of fluorescence intensity was performed using MetaMorph 6.3 software (Molecular Devices/Universal Imaging), as described previously in Sergeev (1996) Endocrine 5: 335-340.

### 8.8.2. Results

Cell Growth and Apoptosis.

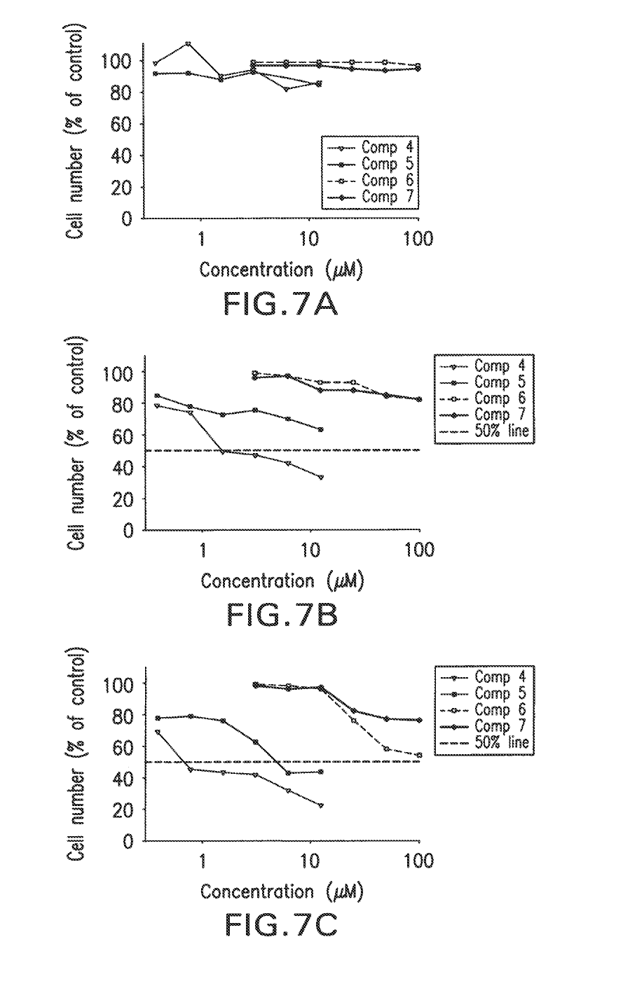

Compounds 4, 5, 6, and 7 inhibited proliferation of MCF-7 breast cancer cells in a dose- and time-dependent fashion, as evaluated by counting cell numbers and determining an increase in the cellular total nucleic acid content. FIG. 7 presents cell number data, where cell numbers of cells treated to PMFs are presented as a percentage of control cells. The IC₅₀ values for inhibition of cell proliferation are presented in Table 15 in which the concentration inhibiting cell growth by 50%, IC₅₀, is presented as the average of the day 3 and day 6 determinations. The apparent minimal effective concentrations, ECₘᵢₙ, for induction of cell death, apoptosis, and increase in [Ca²⁺]ᵢ are also indicated.

TABLE 15
Effective Concentrations of PMFs in MCF-7 Breast Cancer Cells

| | Inhibition | | | |
|---|---|---|---|---|
| | of cell | Induction of | Induction of | Increase in |
| | proliferation | cell apoptosis | cell death | [Ca²⁺]ᵢ |
| Compound | (IC₅₀, μM) | (ECₘᵢₙ, μM) | (ECₘᵢₙ, μM) | (ECₘᵢₙ, μM) |
| 4 | 2.50 | 1.56 | 3.125 | 1.56 |
| 5 | 10.5 | 3.125 | 6.25 | 3.125 |
| 6 | >50 | >50 | >50 | >50 |
| 7 | >50 | >50 | >50 | >50 |

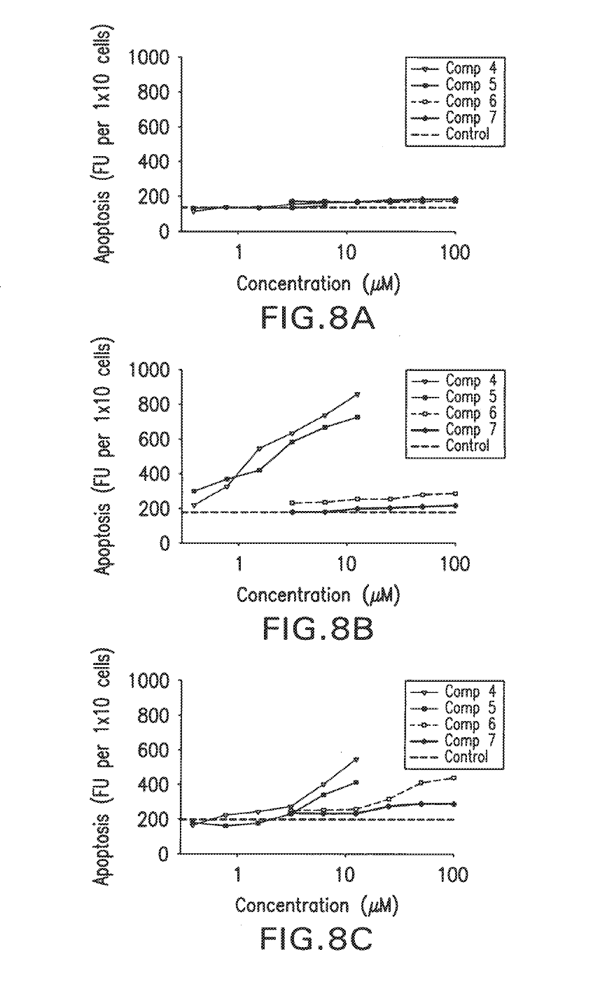

Relative efficacy of the tested compounds for the antiproliferative activity ranked as follows: 4>5>>6>7. Non-hydroxylated compounds 6 and 7 exhibited a very low antiproliferative activity in comparison with hydroxylated compounds 4 and 5. The tested compounds did not exert the cytotoxic effect, as evident by no changes in the numbers of viable cells after a 1-day treatment (see FIG. 8).

Compounds 4 and 5 induced apoptosis in MCF-7 breast cancer cells at day 3 and 6 of treatment in a dose-dependent manner as measured with the fluorescent probe ALEXA FLUOR 488 Annexin V. See FIGS. 8A, B. Effective concentrations were similar to those for the antiproliferative activity (see Table 15). Compound 6 demonstrated low proapototic activity at higher concentrations (25-100 μM) at day 6 of treatment. Compound 7 showed only a trend in inducing apoptosis at day 6 of treatment (50-100 μM). Morphological criteria (nuclear fragmentation) confirmed apoptosis in the cells treated with compounds 4, 5 and 6, as discussed below.

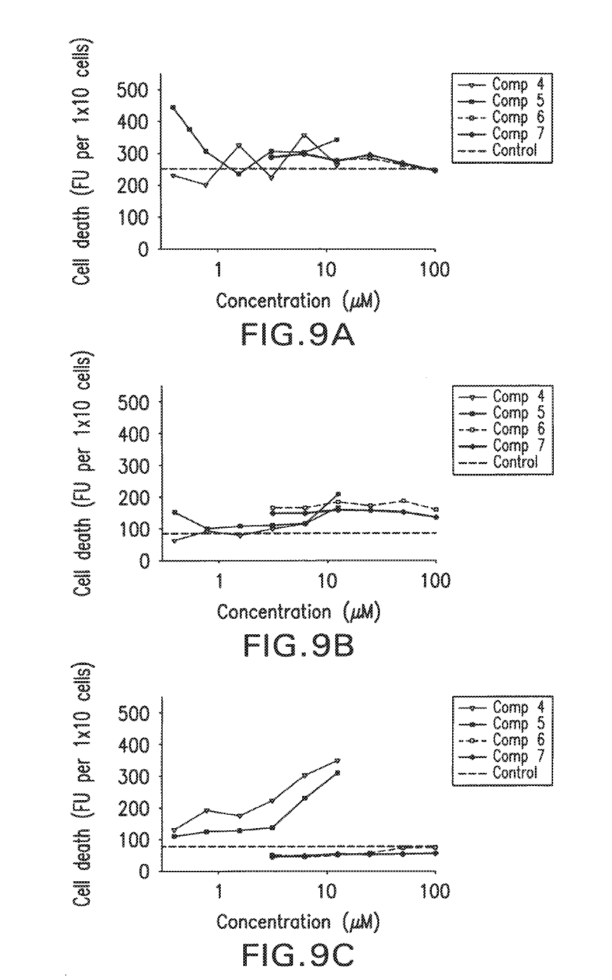

Hydroxylated compounds 4 and 5 induced death of MCF-7 breast cancer cells as evaluated with the propidium iodide. See FIG. 9. The elevated basal fluorescence intensity at day 1 (FIG. 9A) indicates higher permeability of the plasma membrane to propidium iodide of cells recovering after trypsinization. Cell death was evident at day 6 of treatment for PMFs. Non-hydroxylated compound 6 and 7 were not effective in inducing non-apoptotic cell death.

Intracellular Ca²⁺.

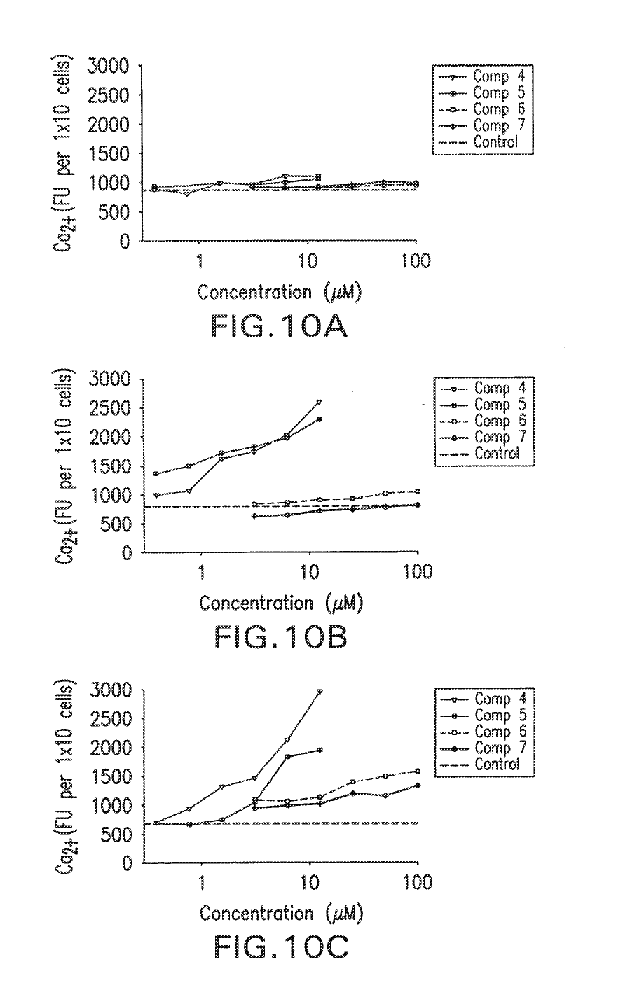

The effects of PMFs on intracellular Ca²⁺ levels in MCF-7 cells. [Ca²⁺]ᵢ was measured with fluo-3/AM, as described above. See FIG. 10. Hydroxylated compounds 4 and 5 induced an increase in the basal level of [Ca²⁺]ᵢ in breast cancer cells at day 3 and 6 of treatment in a dose-dependent fashion. Effective concentrations were similar to those for the antiproliferative and proapoptotic activities (see Table 15). Non-hydroxylated compounds 6 and 7 showed only a trend in increasing intracellular Ca²⁺ levels at higher concentrations (50-100 μM) at day 6 (FIG. 10C) of treatment.

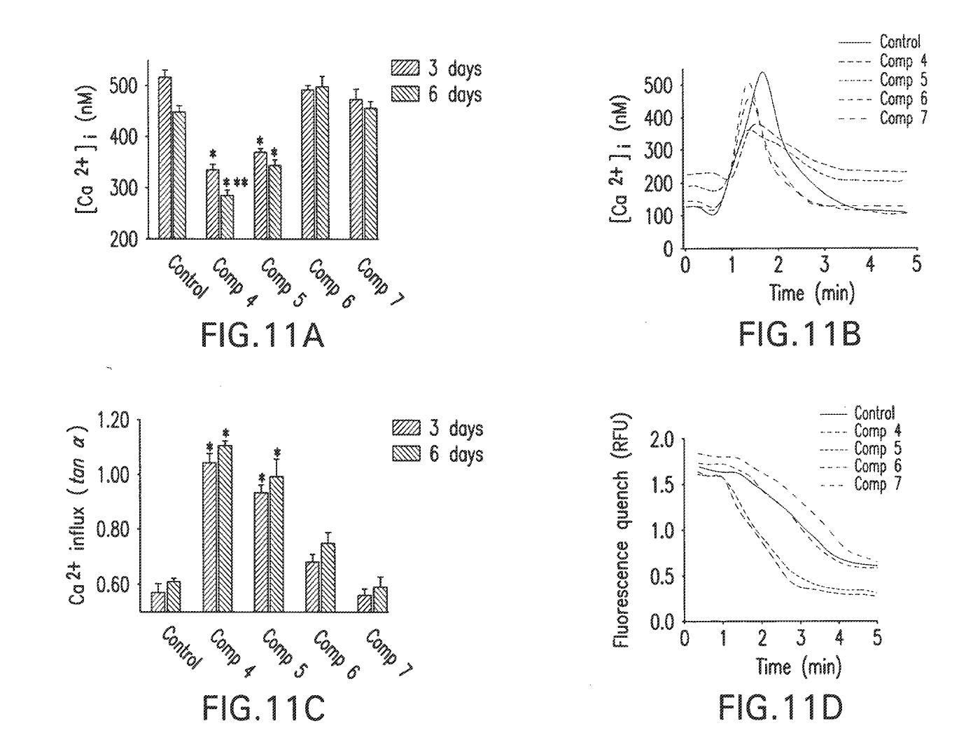

To investigate mechanism of a sustained increase in [Ca²⁺]ᵢ after treatment with PMFs, the rates of the background Ca²⁺ influx from the extracellular space and the magnitudes of Ca²⁺ release from the endoplasmic reticulum stores with thapsigargin were measured in the treated cells using methods described above. Results are shown in FIG. 11.

In FIG. 11A through 11D, the concentration for each PMF was as follows: compound 4: 6.25 μM; compound 5: 12.5 μM; compound 6: 25 μM; and compound 7: 25 μM. The Ca²⁺ mobilization responses presented as a means±SE for the maximum [Ca²⁺]ᵢ rises after addition of thapsigargin in control cells or cells treated with PMFs for 3 days (black bars) or 6 days (gray bars) is depicted in panel A. Panel B shows representative traces of single cell recordings of the Ca²⁺ influx for cells not treated or treated with the compounds. The Ca²⁺ entry rates (C, D) are presented as tangents of the linear portions of the fura-2 quench curves. Data in panel C are presented as means±SE for control cells or cells treated with PMFs 3 days (black bars) or 6 days (gray bars). Panel D shows representative traces of the single cell recordings of Ca²⁺ mobilization. Mn²⁺ or thapsigargin was added after recording the basal level of fluorescence or basal [Ca²⁺]ᵢ for 30-60 s.

The Ca²⁺ mobilization response was significantly decreased in cells treated with hydroxylated compounds 4 and 5 (1.5- and 1.35-fold, respectively), indicating that the tested compounds did deplete intracellular Ca²⁺ stores. FIGS. 11A, B. The smaller Ca²⁺ mobilization response of compound 4 at day 6 of treatment, as compared with the response at day 3, implies that the Ca²⁺ stores were chronically depleted at the later point. Compounds 6 and 7 did not affect the filling of the endoplasmic reticulum Ca²⁺ stores.

Non-hydroxylated compounds 6 and 7 did not significantly increase Ca²⁺ influx, while compounds 4 and 5 markedly increased Ca²⁺ influx (1.8- and 1.6-fold, respectively), as evaluated by the Ca²⁺ entry rates via the low-conductance Ca²⁺ channels. FIGS. 11C,D. The return of [Ca²⁺]ᵢ to basal levels after treatment with thapsigargin was slower in cells treated with compounds 4 and 5 than in controls and cells treated with compounds 6 and 7 (see FIG. 11B). The prolonged “[Ca²⁺]ᵢ decreasing” phase of the response to thapsigargin in Ca²⁺-containing solutions indicates Ca²⁺ entry, consistent with the observation that compounds 4 and 5 increase Ca²⁺ influx.

These findings indicate that an elevated [Ca²⁺]ᵢ in the cells treated with hydroxylated compounds 4 and 5 results from both Ca²⁺ influx from the extracellular space and depletion of the intracellular Ca²⁺ stores.

Calpain and Caspase-12.

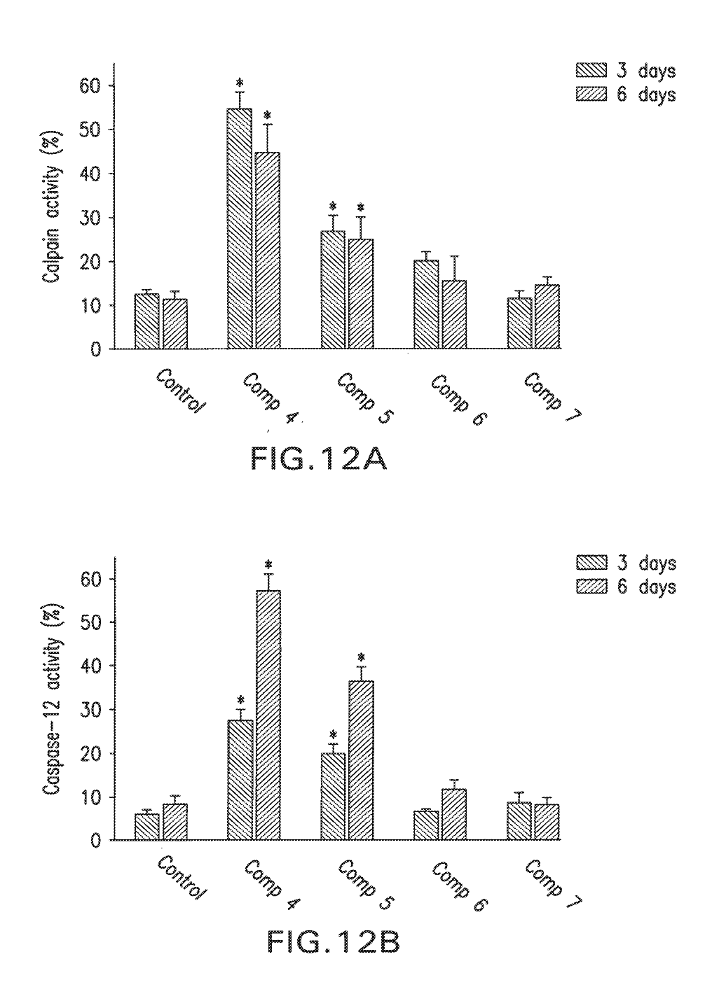

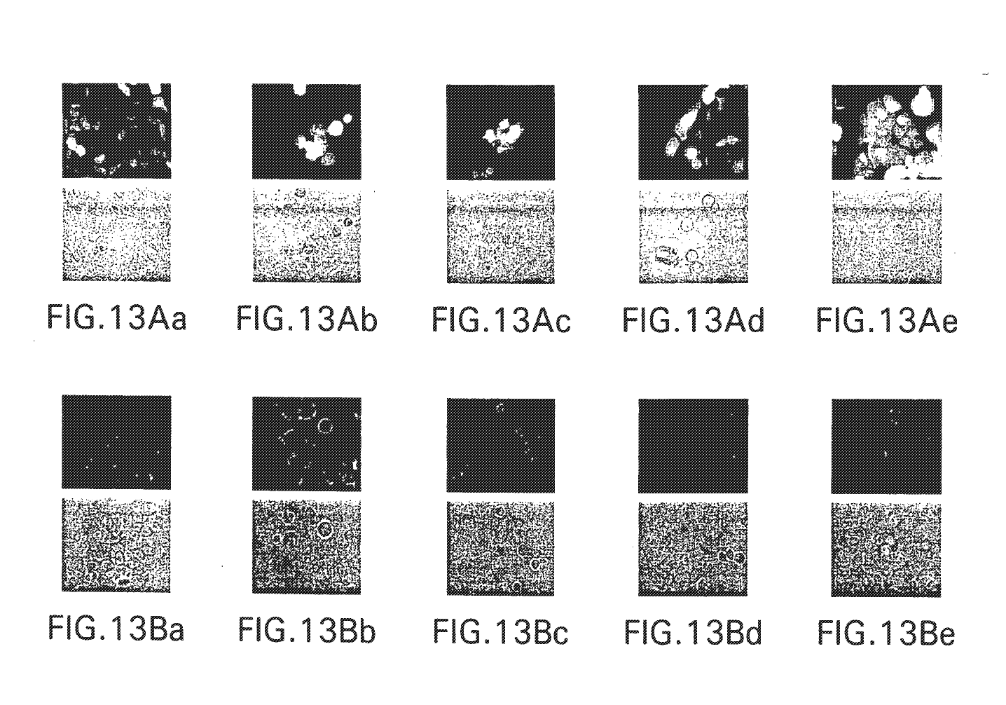

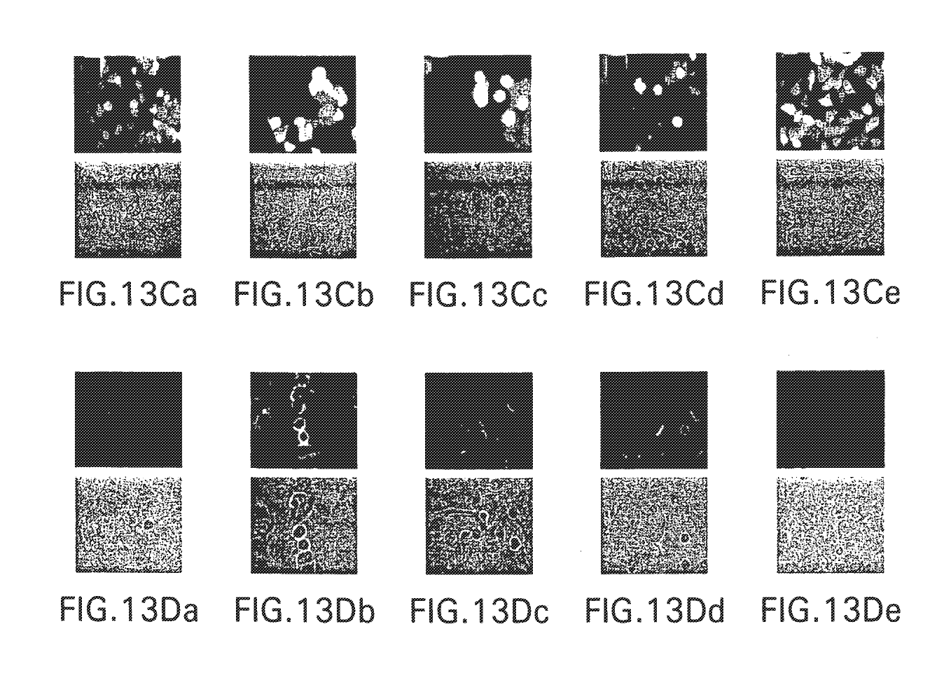

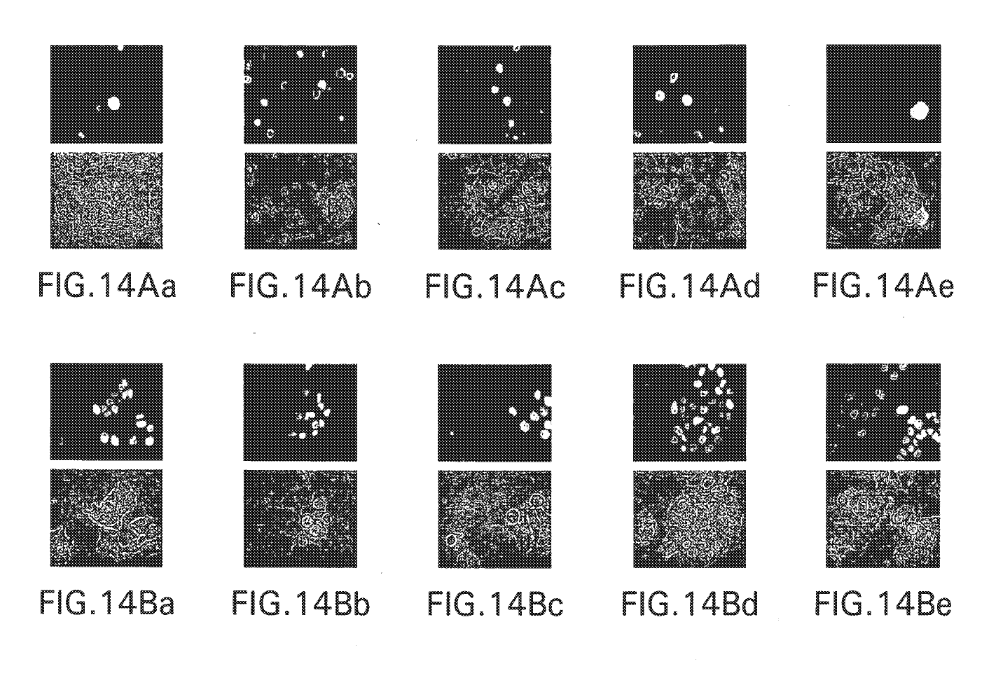

A sustained increase in [Ca²⁺]ᵢ in MCF-7 cells treated with hydroxylated compounds 4 and 5 was accompanied by activation of the Ca²⁺-dependent apoptotic proteases, μ-calpain and caspase-12. The calpain activation was demonstrated by cleavage of the fluorogenic peptide and calpain substrate t-Boc-Leu-Met (FIG. 12A and FIG. 13A) and the presence of the calpain small subunit in the cells (FIG. 13B). Caspase-12 activation in cells treated with compounds 4 and 5 was observed with the peptide substrate ATAD (FIG. 12B and FIG. 13C) and demonstrated with the monoclonal antibodies recognizing truncated caspase-12 (FIG. 13D). No significant calpain and caspase-12 activation was detected in cells treated with non-hydroxylated compounds 6 and 7, although compound 6 demonstrated a trend in increasing the number of cells with activated calpain at day 3 of treatment (see FIGS. 12A,B and FIGS. 13A,B). Caspase-12 was not expressed in the non-apoptotic MCF-7 cells (see FIGS. 13C,D).

These results imply that hydroxylated compounds 4 and 5 induce Ca²⁺-dependent activation of calpain and caspase-12.

Taken together, the results described above evidence the antiproliferative and proapoptotic activity of polymethoxyflavones in breast cancer cells. These data support that PMF-induced sustained increase in [Ca²⁺]ᵢ is associated with induction of apoptosis in these cells and imply that induction of apoptosis with PMFs requires activation of the Ca²⁺-dependent μ-calpain and the Ca²⁺/calpain-dependent caspase-12. Moreover, the results indicate that hydroxylations of PMFs are critical for enhancing their proapoptotic activity.

### 8.9. Example 9

This example provides an exemplary manufacturing process for preparing a composition enriched for hydroxylated PMFs.

Acid Hydrolysis Treatment.

Orange peel extract (OPE) (50 g) containing a 30% PMF fraction was dissolved in 95% ethanol (50 mL) in a 500 mL round bottom flask. 6N HCl (50 mL) was added in gently while stirring. After 5 minutes, the flask was placed in a 105° C. oil bath and the OPE sample was refluxed for 18 hr.

Neutralization.

After the acid-hydrolyzed OPE (AH-OPE) cooled to room temperature, the volume of AH-OPE was reduced in a rotor evaporator with a water bath set at 50° C. After no more liquid condensed from condenser, AH-OPE was neutralized with 3N NaOH. The final pH was around 6 as tested by pH paper. Water (50 mL) was added to AH-OPE before a successive solvent extraction.

### Solvent Extractions:

1. Hexanes Extraction.

Hexanes (50 mL) was added to aqueous AH-OPE and mixed, and the mixture poured into a separatory funnel. Liquid layers containing the hexanes fraction (AH-OPE-Hx) and aqueous AH-OPE fraction were separated and collected. This process was repeated three times. A sample of the combined hexanes fractions, AH-OPE-Hx, was saved for analysis, and the remainder of AH-OPE-Hx was discarded. The aqueous AH-OPE fraction was used in the next extraction step.

2. Ethyl Acetate Extraction.

After hexanes extraction, 50 mL ethyl acetate was added to AH-OPE and mixed. The same procedure was repeated as mentioned above using ethyl acetate in place of hexanes, and an ethyl acetate fraction (AH-OPE-Ea) and an aqueous fraction were separately collected.

Analysis of the OPE, AH-OPE-Hx and AH-OPE-Ea fractions for polymethoxyflavones (PMFs) and hydroxylated-polymethoxyflavones (OH-PMFs) was performed as explained below.

### Results:

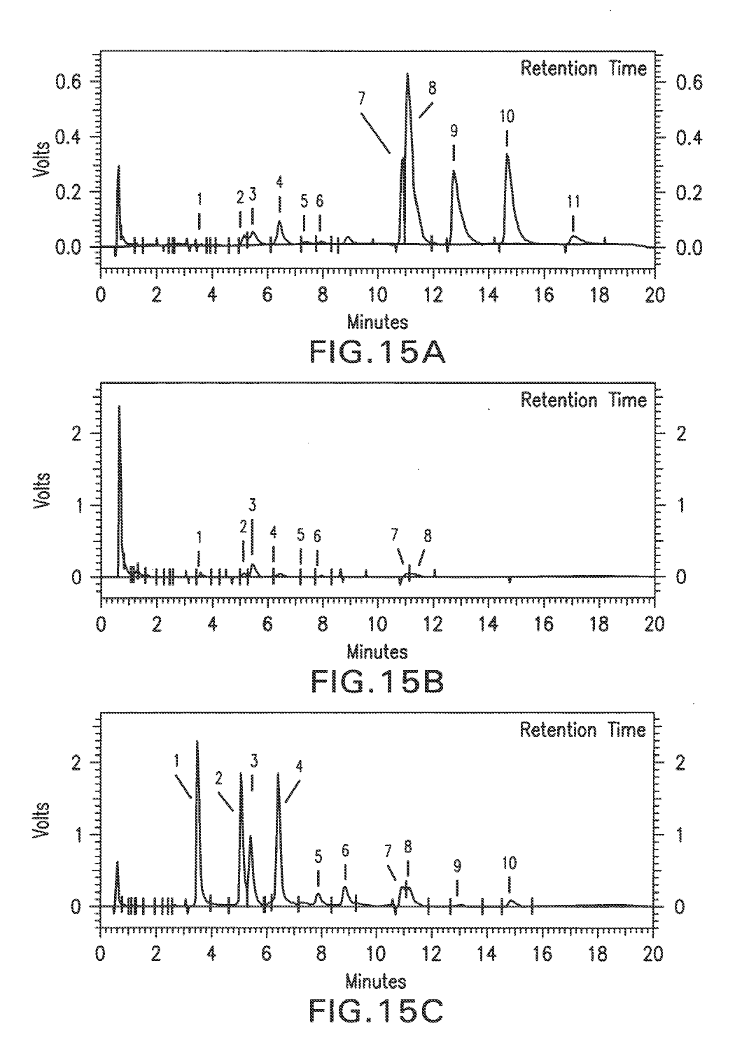

The OPE, AH-OPE-Hx and AH-OPE-Ea fractions described above were subjected to HPLC following a protocol similar to that described in Example 2, above, using UV absorbance at 280 nm to assess relative concentrations for PMFs and OH-PMFs in the fractions. The chromatograms shown in FIG. 15 correspond to those of OPE (FIG. 15A), AH-OPE-Hx (FIG. 15B), and AH-OPE-Ea (FIG. 15B). Peaks numbered 1-11 in the chromatograms shown in FIG. 15A-C correspond to the following OH-PMFs and PMFs shown in Table 16.

TABLE 16

| Peak | | |
|---|---|---|
| No. | Type | PMF |
| 1 | hydroxylated | 5-Hydroxy-6,7,8,4′-tetramethoxyflavone |
| | | (5-demethyltangeretin) |
| 2 | hydroxylated | 5-Hydroxy-6,7,4′-trimethoxyflavone |
| 3 | hydroxylated | 5-Hydroxy-3,6,7,8,3′,4′-hexamethoxyflavone |
| 4 | hydroxylated | 5-Hydroxy-6,7,8,3′,4′-pentamethoxyflavone |
| | | (5-Demethylnobiletin) |
| 5 | hydroxylated | 5-Hydroxy-3,6,7,3′,4′-pentamethoxyflavone |
| 6 | hydroxylated | 5-Hydroxy-6,7,3′,4′-tetramethoxyflavone |
| 7 | non-hydroxylated | Heptamethoxyflavone |
| 8 | non-hydroxylated | Tangeretin |
| 9 | non-hydroxylated | 5,6,7,4′-Tetramethoxyflavone |
| 10 | non-hydroxylated | Nobiletin |
| 11 | non-hydroxylated | Sinesetin |

For each chromatogram shown in FIG. 15, 15 μl of sample was loaded where the concentrations of the fractions were as follows: OPE, 12.85 mg/mL; AH-OPE-Hx, 25.40 mg/mL; AH-OPE-Ea, 11.20 mg/mL. As shown in FIG. 15A, the OPE starting material had a relatively large non-hydroxylated PMF component (peaks 7-11) compared to the hydroxylated component (peaks 1-6). The method of acidifying and extracting the starting material described above was very effective in producing a hydroxylated PMF-enriched fraction (peaks 1-6 in AH-OPE-Ea, FIG. 15C), with relatively little loss during removal of non-PMFs (AH-OPE-Hx, FIG. 15B).

All publications, patents and patent applications mentioned in this specification are herein incorporated by reference into the specification to the same extent as if each individual publication, patent or patent application was specifically and individually indicated to be incorporated herein by reference. Citation or discussion of a reference herein shall not be construed as an admission that such is prior art to the present invention.

While the invention has been described in terms of various preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

1. A plant extract composition enriched with hydroxylated polymethoxyflavones comprising a polymethoxyflavone (PMF) fraction having between 15% (w/w) and 95% (w/w) of one or more hydroxylated polymethoxyflavones, wherein the proportion of hydroxylated polymethoxyflavones to non-hydroxylated polymethoxyflavones in the plant extract composition is greater than the proportion of hydroxylated polymethoxyflavones to non-hydroxylated polymethoxyflavones found naturally in the plant from which the extract is derived, and wherein the composition comprises at least one hydroxylated PMF selected from the group consisting of
3-hydroxy-5,6,7,4′-tetramethoxyflavone,
3-hydroxy-5,6,7,8,4′-pentamethoxyflavone,
3-hydroxy-5,6,7,8,3′,4′-hexamethoxyflavone,
5-hydroxy-3,6,7,3′,4′-pentamethoxyflavone,
5-hydroxy-3,7,8,3′,4′-pentamethoxyflavone,
5-hydroxy-3,7,3′,4′-tetramethoxyflavone,
5-hydroxy-6,7,8,3′,4′,5′-hexamethoxyflavone,
5-hydroxy-6,7,3′,4′-tetramethoxyflavone,
5-hydroxy-6,7,4′-trimethoxyflavone,
5,3′-dihydroxy-6,7,8,4′-tetramethoxyflavone,
3′-hydroxy-5,6,7,4′-tetramethoxyflavone,
3′-hydroxy-5,6,7,8,4′-pentamethoxyflavone,
3′,4′-dihydroxy-5,6,7,8-tetramethoxyflavone, and
4′-hydroxy-5,6,7,8,3′-pentamethoxyflavone.

2. The plant extract of claim 1, wherein the PMF fraction comprises between about 50% (w/w) to about 95% (w/w) of one or more hydroxylated PMFs.

3. The plant extract of claim 1, wherein the PMF fraction comprises between about 50% (w/w) to about 90% (w/w) of one or more hydroxylated PMFs.

4. The plant extract of claim 1, wherein the PMF fraction comprises between about 50% (w/w) to about 85% (w/w) of one or more hydroxylated PMFs.

5. The plant extract of claim 1, wherein the PMF fraction further comprises one or more non-hydroxylated PMFs.

6. The plant extract of claim 1, wherein the one or more hydroxylated PMFs consist of hydroxylated PMFs selected from the group consisting of
3-hydroxy-5,6,7,4′-tetramethoxyflavone,
3-hydroxy-5,6,7,8,4′-pentamethoxyflavone,
3-hydroxy-5,6,7,8,3′,4′-hexamethoxyflavone,
5-hydroxy-3,6,7,3′,4′-pentamethoxyflavone,
5-hydroxy-3,6,7,8,3′,4′-hexamethoxyflavone,
5-hydroxy-3,7,8,3′,4′-pentamethoxyflavone,
5-hydroxy-3,7,3′,4′-tetramethoxyflavone,
5-hydroxy-6,7,8,3′,4′-pentamethoxyflavone,
5-hydroxy-6,7,8,3′,4′,5′-hexamethoxyflavone,
5-hydroxy-6,7,8,4′-tetramethoxyflavone,
5-hydroxy-6,7,3′,4′-tetramethoxyflavone,
5-hydroxy-6,7,4′-trimethoxyflavone,
5,3′-dihydroxy-6,7,8,4′-tetramethoxyflavone,
5-hydroxy-7,8,3′,4′-tetramethoxyflavone,
5,7-dihydroxy-6,8,3′,4′-tetramethoxyflavone,
7-hydroxy-3,5,6,8,3′,4′-hexamethoxyflavone,
7-hydroxy-3,5,6,3′,4′-pentamethoxyflavone,
3′-hydroxy-5,6,7,4′-tetramethoxyflavone,
3′-hydroxy-5,6,7,8,4′-pentamethoxyflavone,
3′,4′-dihydroxy-5,6,7,8-tetramethoxyflavone and
4′-hydroxy-5,6,7,8,3′-pentamethoxyflavone.

7. A composition enriched with hydroxylated polymethoxyflavones comprising between at least 15% (w/w) and 95% (w/w) hydroxylated polymethoxyflavones (PMFs), wherein the proportion of hydroxylated polymethoxyflavones to non-hydroxylated polymethoxyflavones in the composition is greater than the proportion of hydroxylated polymethoxyflavones to non-hydroxylated polymethoxyflavones found naturally in the source from which the PMFs are derived, and wherein the composition comprises at least one hydroxylated PMF selected from the group consisting of
3-hydroxy-5,6,7,4′-tetramethoxyflavone,
3-hydroxy-5,6,7,8,4′-pentamethoxyflavone,
3-hydroxy-5,6,7,8,3′,4′-hexamethoxyflavone,
5-hydroxy-3,6,7,3′,4′-pentamethoxyflavone,
5-hydroxy-3,7,8,3′,4′-pentamethoxyflavone,
5-hydroxy-3,7,3′,4′-tetramethoxyflavone,
5-hydroxy-6,7,8,3′,4′,5′-hexamethoxyflavone,
5-hydroxy-6,7,3′,4′-tetramethoxyflavone,
5-hydroxy-6,7,4′-trimethoxyflavone,
5,3′-dihydroxy-6,7,8,4′-tetramethoxyflavone,
3′-hydroxy-5,6,7,4′-tetramethoxyflavone,
3′-hydroxy-5,6,7,8,4′-pentamethoxyflavone,
3′,4′-dihydroxy-5,6,7,8-tetramethoxyflavone, and
4′-hydroxy-5,6,7,8,3′-pentamethoxyflavone.

8. The composition of claim 7, which is a plant extract composition.

9. The plant extract composition of claim 8, wherein the composition comprises between about 20% (w/w) and about 90% (w/w) hydroxylated PMFs.

10. The plant extract composition of claim 8, wherein the composition comprises between about 40% (w/w) and about 85% (w/w) hydroxylated PMFs.

11. The plant extract composition of claim 9, wherein the plant extract composition is an orange peel extract.

12. The plant extract composition of claim 9, wherein the plant extract composition is a dietary supplement, food additive or nutraceutical.

13. The plant extract composition of claim 9, wherein the plant extract composition is a cosmetic composition.

14. The plant extract composition of claim 11, wherein the hydroxylated PMFs comprise at least two hydroxylated PMFs selected from the group consisting of
3-hydroxy-5,6,7,4′-tetramethoxyflavone,
3-hydroxy-5,6,7,8,4′-pentamethoxyflavone,
3-hydroxy-5,6,7,8,3′,4′-hexamethoxyflavone,
5-hydroxy-3,6,7,3′,4′-pentamethoxyflavone,
5-hydroxy-3,6,7,8,3′,4′-hexamethoxyflavone,
5-hydroxy-3,7,8,3′,4′-pentamethoxyflavone,
5-hydroxy-3,7,3′,4′-tetramethoxyflavone,
5-hydroxy-6,7,8,3′,4′-pentamethoxyflavone,
5-hydroxy-6,7,8,3′,4′,5′-hexamethoxyflavone,
5-hydroxy-6,7,8,4′-tetramethoxyflavone,
5-hydroxy-6,7,3′,4′-tetramethoxyflavone,
5-hydroxy-6,7,4′-trimethoxyflavone,
5,3′-dihydroxy-6,7,8,4′-tetramethoxyflavone,
5-hydroxy-7,8,3′,4′-tetramethoxyflavone,
5,7-dihydroxy-6,8,3′,4′-tetramethoxyflavone,
7-hydroxy-3,5,6,8,3′,4′-hexamethoxyflavone,
7-hydroxy-3,5,6,3′,4′-pentamethoxyflavone,
3′-hydroxy-5,6,7,4′-tetramethoxyflavone,
3′-hydroxy-5,6,7,8,4′-pentamethoxyflavone,
3′,4′-dihydroxy-5,6,7,8-tetramethoxyflavone, and
4′-hydroxy-5,6,7,8,3′-pentamethoxyflavone.

15. The plant extract composition of claim 11, wherein the hydroxylated PMFs consist essentially of two hydroxylated PMFs selected from the group consisting of
3-hydroxy-5,6,7,4′-tetramethoxyflavone,
3-hydroxy-5,6,7,8,4′-pentamethoxyflavone,
3-hydroxy-5,6,7,8,3′,4′-hexamethoxyflavone,
5-hydroxy-3,6,7,3′,4′-pentamethoxyflavone,
5-hydroxy-3,6,7,8,3′,4′-hexamethoxyflavone,
5-hydroxy-3,7,8,3′,4′-pentamethoxyflavone,
5-hydroxy-3,7,3′,4′-tetramethoxyflavone,
5-hydroxy-6,7,8,3′4′-pentamethoxyflavone,
5-hydroxy-6,7,8,3′,4′,5′-hexamethoxyflavone,
5-hydroxy-6,7,8,4′-tetramethoxyflavone,
5-hydroxy-6,7,3′,4′-tetramethoxyflavone,
5-hydroxy-6,7,4′-trimethoxyflavone,
5,3′-dihydroxy-6,7,8,4′-tetramethoxyflavone,
5-hydroxy-7,8,3′,4′-tetramethoxyflavone,
5,7-dihydroxy-6,8,3′,4′-tetramethoxyflavone,
7-hydroxy-3,5,6,8,3′,4′-hexamethoxyflavone,
7-hydroxy-3,5,6,3′,4′-pentamethoxyflavone,
3′-hydroxy-5,6,7,4′-tetramethoxyflavone,
3′-hydroxy-5,6,7,8,4′-pentamethoxyflavone,
3′,4′-dihydroxy-5,6,7,8-tetramethoxyflavone, and
4′-hydroxy-5,6,7,8,3′-pentamethoxyflavone.

16. The plant extract composition of claim 15, wherein the at least two hydroxylated PMFs are 3′-hydroxy-5,6,7,4′-tetramethoxyflavone and 5-hydroxy-3,6,7,8,3′,4′-hexamethoxyflavone.

17. The plant extract composition of claim 11, wherein the hydroxylated PMFs comprise at least three hydroxylated PMFs selected from the group consisting of
3-hydroxy-5,6,7,4′-tetramethoxyflavone,
3-hydroxy-5,6,7,8,4′-pentamethoxyflavone,
3-hydroxy-5,6,7,8,3′,4′-hexamethoxyflavone,
5-hydroxy-3,6,7,3′,4′-pentamethoxyflavone,
5-hydroxy-3,6,7,8,3′,4′-hexamethoxyflavone,
5-hydroxy-3,7,8,3′,4′-pentamethoxyflavone,
5-hydroxy-3,7,3′,4′-tetramethoxyflavone,
5-hydroxy-6,7,8,3′,4′-pentamethoxyflavone,
5-hydroxy-6,7,8,3′,4′,5′-hexamethoxyflavone,
5-hydroxy-6,7,8,4′-tetramethoxyflavone,
5-hydroxy-6,7,3′4′-tetramethoxyflavone,
5-hydroxy-6,7,4′-trimethoxyflavone,
5,3′-dihydroxy-6,7,8,4′-tetramethoxyflavone,
5-hydroxy-7,8,3′,4′-tetramethoxyflavone,
5,7-dihydroxy-6,8,3′,4′-tetramethoxyflavone,
7-hydroxy-3,5,6,8,3′,4′-hexamethoxyflavone,
7-hydroxy-3,5,6,3′,4′-pentamethoxyflavone,
3′-hydroxy-5,6,7,4′-tetramethoxyflavone,
3′-hydroxy-5,6,7,8,4′-pentamethoxyflavone,
3′,4′-dihydroxy-5,6,7,8-tetramethoxyflavone, and
4′-hydroxy-5,6,7,8,3′-pentamethoxyflavone.

18. The plant extract composition of claim 11, wherein the hydroxylated PMFs comprise at least four hydroxylated PMFs selected from the group consisting of
3-hydroxy-5,6,7,4′-tetramethoxyflavone,
3-hydroxy-5,6,7,8,4′-pentamethoxyflavone,
3-hydroxy-5,6,7,8,3′,4′-hexamethoxyflavone,
5-hydroxy-3,6,7,3′,4′-pentamethoxyflavone,
5-hydroxy-3,6,7,8,3′,4′-hexamethoxyflavone,
5-hydroxy-3,7,8,3′,4′-pentamethoxyflavone,
5-hydroxy-3,7,3′,4′-tetramethoxyflavone,
5-hydroxy-6,7,8,3′,4′-pentamethoxyflavone,
5-hydroxy-6,7,8,3′,4′,5′-hexamethoxyflavone,
5-hydroxy-6,7,8,4′-tetramethoxyflavone,
5-hydroxy-6,7,3′,4′-tetramethoxyflavone,
5-hydroxy-6,7,4′-trimethoxyflavone,
5,3′-dihydroxy-6,7,8,4′-tetramethoxyflavone,
5-hydroxy-7,8,3′,4′-tetramethoxyflavone,
5,7-dihydroxy-6,8,3′,4′-tetramethoxyflavone,
7-hydroxy-3,5,6,8,3′,4′-hexamethoxyflavone,
7-hydroxy-3,5,6,3′,4′-pentamethoxyflavone,
3′-hydroxy-5,6,7,4′-tetramethoxyflavone,
3′-hydroxy-5,6,7,8,4′-pentamethoxyflavone,
3′,4′-dihydroxy-5,6,7,8-tetramethoxyflavone, and
4′-hydroxy-5,6,7,8,3′-pentamethoxyflavone.

19. The plant extract composition of claim 11, wherein the hydroxylated PMFs comprise at least five hydroxylated PMFS selected from the group consisting of
3-hydroxy-5,6,7,4′-tetramethoxyflavone,
3-hydroxy-5,6,7,8,4′-pentamethoxyflavone,
3-hydroxy-5,6,7,8,3′,4′-hexamethoxyflavone,
5-hydroxy-3,6,7,3′,4′-pentamethoxyflavone,
5-hydroxy-3,6,7,3,3′,4′-hexamethoxyflavone,
5-hydroxy-3,7,8,3′,4′-pentamethoxyflavone,
5-hydroxy-3,7,3′,4′-tetramethoxyflavone,
5-hydroxy-6,7,8,3′,4′-pentamethoxyflavone,
5-hydroxy-6,7,8,3′,4′,5′-hexamethoxyflavone,
5-hydroxy-6,7,8,4′-tetramethoxyflavone,
5-hydroxy-6,7,3′,4′-tetramethoxyflavone,
5-hydroxy-6,7,4′-trimethoxyflavone,
5,3′-dihydroxy-6,7,8,4′-tetramethoxyflavone,
5-hydroxy-7,8,3′,4′-tetramethoxyflavone,
5,7-dihydroxy-6,8,3′,4′-tetramethoxyflavone,
7-hydroxy-3,5,6,8,3′,4′-hexamethoxyflavone,
7-hydroxy-3,5,6,3′,4′ pentamethoxyflavone,
3′-hydroxy-5,6,7,4′-tetramethoxyflavone,
3′-hydroxy-5,6,7,8,4′-pentamethoxyflavone,
3′,4′-dihydroxy-5,6,7,8-tetramethoxyflavone, and
4′-hydroxy-5,6,7,8,3′-pentamethoxyflavone.

20. The plant extract composition of claim 11, wherein the hydroxylated PMFs comprise at least six hydroxylated PMFs selected from the group consisting of
3-hydroxy-5,6,7,4′-tetramethoxyflavone,
3-hydroxy-5,6,7,8,4′-pentamethoxyflavone,
3-hydroxy-5,6,7,8,3′,4′-hexamethoxyflavone,
5-hydroxy-3,6,7,3′,4′-pentamethoxyflavone,
5-hydroxy-3,6,7,8,3′,4′-hexamethoxyflavone,
5-hydroxy-3,7,8,3′,4′-pentamethoxyflavone,
5-hydroxy-3,7,3′,4′-tetramethoxyflavone,
5-hydroxy-6,7,8,3′,4′-pentamethoxyflavone,
5-hydroxy-6,7,8,3′,4′,5′-hexamethoxyflavone,
5-hydroxy-6,7,8,4′-tetramethoxyflavone,
5-hydroxy-6,7,3′,4′-tetramethoxyflavone,
5-hydroxy-6,7,4′-trimethoxyflavone,
5,3′-dihydroxy-6,7,8,4′-tetramethoxyflavone,
5-hydroxy-7,8,3′,4′-tetramethoxyflavone,
5,7-dihydroxy-6,8,3′,4′-tetramethoxyflavone,
7-hydroxy-3,5,6,8,3′,4′-hexamethoxyflavone,
7-hydroxy-3,5,6,3′,4′-pentamethoxyflavone,
3′-hydroxy-5,6,7,4′-tetramethoxyflavone,
3′-hydroxy-5,6,7,8,4′-pentamethoxyflavone,
3′,4′-dihydroxy-5,6,7,8-tetramethoxyflavone, and
4′-hydroxy-5,6,7,8,3′-pentamethoxyflavone.

21. The plant extract composition of claim 9, further comprising a member selected from the group consisting of
5-hydroxy-6,7,8,3′,4′-pentamethoxyflavanone,
2′-hydroxy-3,4,4′,5′,6′-pentamethoxychalcone and
2′-hydroxy-3,4,3′,4′,5′,6′-pentamethoxychalcone.

22-54. (canceled)
